(19) United States
(12) Patent Application Publication
(10) Pub. No.: US 2025/0318731 A1
(43) Pub. Date: Oct. 16, 2025

(54) SYSTEM AND METHOD FOR NON-INVASIVE ASSESSMENT AND TREATMENT OF INFLAMMATORY CONDITIONS

(71) Applicant: NIMBIO LTD, Tel-Mond (IL)

(72) Inventor: Tal ATAROT, Tel-Mond (IL)

(73) Assignee: NIMBIO LTD, Tel-Mond (IL)

(21) Appl. No.: 18/698,290

(22) PCT Filed: Sep. 22, 2022
(86) PCT No.: PCT/IL2022/051021

§ 371 Date: Apr. 3, 2024

Related U.S. Application Data
(60) Provisional application No. 63/252,283, filed on Oct. 5, 2021.

(51) Int. Cl. A61B 5/00; A61B 5/02; A61B 5/024; A61B 5/0295; A61B 5/11; A61B 5/145; A61B 5/1455

(52) U.S. Cl. CPC A61B 5/0053; A61B 5/0051; A61B 5/0057; A61B 5/02007; A61B 5/024; A61B 5/0295; A61B 5/1126; A61B 5/14535; A61B 5/14546; A61B 5/1455; A61B 5/4806; A61B 5/6815; A61B 5/6823; A61B 5/6824; A61B 5/6826; A61B 5/6828; A61B 5/6829; A61B 5/683; A61B 5/7246; A61B 5/7264; A61B 5/7275; A61B 5/7285; A61B 2560/0242; A61B 2562/0204; A61B 2562/0219; A61B 2562/0238; A61B 2562/0271; A61B 2562/029

(57) ABSTRACT

The present disclosure provides methods and systems for monitoring of physiological parameters of a patient. More specifically, the present disclosure provides non-invasive assessment and treatment of inflammatory conditions in patients.

### FIELD OF THE INVENTION

The present invention relates generally to monitoring of physiological parameters. More specifically, the present invention relates to non-invasive assessment and treatment of inflammatory conditions and status in patients.

### BACKGROUND OF THE INVENTION

Inflammation is a physiological response to potential danger signals and damage in organs in our body. In diseases such as immune mediated diseases; said immune mediated diseases being selected from a group consisting of Inflammatory Bowel Diseases (IBD) including Ulcerative Colitis (UC) and Crohn's disease (CD), Rheumatological diseases, Rheumatoid Arthritis (RA), Psoriatic Arthritis (PsA), Spondyloarthritis (SpA) Psoriasis, Chronic Obstructive Pulmonary Disease (COPD), Asthma, Systemic Lupus Erythematosus (SLE), Multiple Sclerosis (MS), Vasculitis, Immune-Mediated Heart Diseases, Infection-Related Immune-Mediated Diseases and others, immune system activity is the cause for organ damage.

The inflammatory process is an important function for injury repair and control. Commonly referred to as the inflammatory cascade, or simply inflammation, it can take two basic forms, acute and chronic. Acute inflammation, part of the immune response, is the body's immediate response to injury or assault due to physical trauma, infection, stress, or a combination of all three.

When inflammation becomes self-perpetuating however, it can result in chronic or long-term inflammation. This process is known as chronic inflammation and lasts beyond the actual injury; sometimes for months or years. It can become a problem by itself, and require medical intervention aimed at its control on inflammation-mediated damage.

According to the world health organization (WHO), by 2020 chronic inflammatory diseases are expected to contribute to 73% of all deaths and 60% of economic burden of disease. This makes chronic inflammatory diseases/immune-mediated diseases a major health challenge in modern societies. immune mediated diseases; said immune mediated diseases being selected from a group consisting of Inflammatory Bowel Diseases (IBD) including ulcerative colitis (UC) and Crohn's disease (CD), Rheumatoid Arthritis (RA), Psoriatic Arthritis (PsA), Psoriasis and Chronic Obstructive Pulmonary Disease (COPD), Systemic Lupus Erythematosus (SLE), Multiple Sclerosis (MS), are examples of immune-mediated diseases with significant social and economic burden.

Chronic inflammation can affect any and all body organs. Inflammation can also be a secondary component of many diseases. For example, in atherosclerosis, or arterial damage, where chronic inflammation of blood vessel walls can result in arterial plaque build-up, arterial or vascular blockages, and lead to ischemic heart disease. Chronic inflammation also plays a significant role in other diseases and conditions as well; chronic pain, poor sleep quality, obesity, physical impairment, and overall decreased quality of life.

Chronic Inflammation may also serve as a precursor for certain cancers. Persistent inflammation is associated with DNA damage, which in turn can lead to cancer. For example, people with IBD have an increased risk of colon cancer.

While acute inflammation is a part of the body's natural defence system against injury and disease, chronic inflammation is considered a disease by itself. Since chronic inflammation commonly affects specific organs and may be associated with a defined disease process, treatment approaches vary considerably.

Chronic inflammatory diseases take a huge toll on quality of life of hundreds of millions of people. Late detection and therapeutic solutions have a significant economic burden that can only be expressed in trillions of Dollars. In addition, chronic inflammation often leads to co-development of other diseases. There are no sufficient medical solutions in the market for these diseases. Treatment modalities consist among others of biologic drugs, which need to be applied parenterally. For example, one of the highest selling drugs in the world, Humira®, which treats Rheumatoid Arthritis, Inflammatory bowel diseases etc., has long term efficacy in only 20-25% of the patient population and can have significant side effects.

Steroids are commonly used to suppress immune response. Though an important modality, steroids are associated with common and significant side effects. Because of these side effects, modern, more advanced therapy is based on detailed understanding of immune system activity combined with agents targeted at key point inflammatory factors, of which both biologics and small molecules are used to eliminate their activity.

In addition, therapeutic targets have evolved beyond symptom control and were set to prevent organ damage with an intent to normalize long term organ function. Such advanced therapy aimed at permanent and continuous control of inflammation requires a treatment paradigm shift. Such change includes a personalized treatment approach to best fit and achieve maximal efficacy in each patient. Personal care involves matching the drug to the patient, assuring its proper dosing while carefully monitoring the inflammatory process in order to detect early loss of response (LOR). Such early detection is of major importance as an alert for a need to adjust drug dosage or shift therapeutic modalities to assure the continuous control of inflammation. In addition, early detection of subclinical inflammation is important for increasing treatment efficacy by avoiding the delay enforced by relaying on clinical detection of flares only. Thus, early detection of flares and the effects of a successful intervention preceding clinical improvement are powerful tools to guide and personalize treatment with current drugs in order to improve medical outcomes.

The medical need is therefore to shift delivery of ambulatory care from acute, episodic, and reactive encounters, to proactive, planned, and longitudinal care.

The purpose is to improve quality of care and population health outcomes, while reducing healthcare costs for patients with chronic inflammatory diseases. A reduction in patient symptom intensity which will result in declines in hospital admissions and use of Emergency Rooms.

The best opportunities to achieve the unmet medical need relay in remote disease activity surveillance while optimizing drugs use, preventing avoidable acute care services, controlling disease progression, and ensuring care coordination of patients with high resource utilization.

The need for monitoring inflammatory responses is not limited to chronic inflammatory diseases. Diseases involving acute inflammation merit monitoring of the inflammatory response as well. A prominent example may be infectious diseases. For example. The COVID-19 disease epidemic is characterized by a biphasic disease wherein the first phase is mediated by the actual viral infection and the second phase is characterized by uncontrolled immune response which is associated with elevated CRP, D-Dimer and other acute phase reactants. Due to the contagious risk during delivery of medical care, a mean to detect changes in the inflammatory status while avoiding direct patient contact would be extremely useful. Similarly, continuous monitoring of inflammation may be useful in the context of intensive care units where patient status is unstable, and an early indication of deterioration may allow to shift care accordingly.

The ability to rapidly detect changes in the inflammatory score may have a significant impact of patient medical care. For example, in the case of COVID-19 a shift to the inflammatory phase may merit administration of anti-inflammatory drugs such as glucocorticoids or anti-IL6 therapy such as Tocolizumab. In the case of sepsis patients in the ICU such detection may allow to change or initiate antibiotic therapy or consider anti-fungal treatment in the appropriate settings.

Currently available systems may perform non-invasive assessment medical conditions in patients, based on sensors that may be attached to a patient's body. For example, medical monitoring systems may employ a photoplethysmography (PPG) sensor, to continuously monitor heart rate and oxygen saturation levels.

Recent studies demonstrated that optimizing therapy through proactive disease management involving reaction to elevated inflammatory biomarkers in addition to the clinical complaints, termed “tight control” results in superior treatment outcomes and is eventually cost effective. Significant culprits associated with this approach are patient engagement and available medical resources. Indeed, data suggests that up to 75% of patients needed ER services did not have a prior contact with their primary IBD care giver (see “Effect of tight control management on Crohn's disease (CALM): a multicentre, randomised, controlled phase 3 trial”; Colombel at al, The Lancet 2017; 390:2779-89; http://dx.doi.org/10.1016/S0140-6736 (17) 32641-7). One potential solution to these challenges may be the use a novel personalized “inflammometer” incorporated in a practical remote inflammation-responsive disease management platform containing the relevant medical information to provide a solution to the unmet need of early detection of asymptomatic upcoming flare-ups and treatment failure secondary to drug non-response leading to proactive treatment adjustments and individualized patient management. Wherein patient data acquisition demands minimal effort from the patient and the necessary data for decision making is readily available for the care team in conjunction with readily available medical data. This unmet need in the field of medical diagnostics and treatment to continuously and non-invasively (e.g., without drawing blood) assess and monitor inflammatory conditions and enable timely and optimal remote care in patients remained a great need. Such monitoring may allow healthcare providers to evaluate the efficacy of treatment, and enable rapid intervention in case of an inflammatory flare. Thus, there is still remain a long felt need for such a system to provide optimal remote monitoring and treatment optimization.

### SUMMARY OF THE INVENTION

Recent studies demonstrated that optimizing therapy through proactive disease management involving reaction to elevated inflammatory biomarkers in addition to the clinical complaints, termed “tight control” results in superior treatment outcomes and is eventually cost effective. Significant culprits associated with this approach are patient engagement and available medical resources. Indeed, data suggests that up to 75% of patients needed ER services did not have a prior contact with their primary IBD care giver. One potential solution to these challenges may be the use a novel personalized “inflammometer” incorporated in a practical remote inflammation-responsive disease management platform containing the relevant medical information to provide a solution to the unmet need of early detection of asymptomatic upcoming flare-ups and treatment failure secondary to drug non-response leading to proactive treatment adjustments and individualized patient management, wherein patient data acquisition demands minimal effort from the patient and the necessary data for decision making is readily available for the care team in conjunction with readily available medical data. This unmet need in the field of medical diagnostics and treatment to continuously and non-invasively (e.g., without drawing blood) assess and monitor inflammatory conditions and enable timely and optimal remote care in patients, has intensified due to the COVID19 pandemic. Such monitoring may allow healthcare providers to evaluate the efficacy of treatment, and enable rapid intervention in case of an inflammatory flare. Embodiments of the invention may facilitate these goals by using artificial intelligence and machine learning methodologies, to assess the patient's condition in real time or near real time, and provide efficient treatment.

It is thus one object of the present invention to provide a method of indicating of an inflammatory status in a patient, comprising steps of:
receiving at least one photoplethysmography (PPG) signal from at least one location pertaining to the patient; and,
analyzing said at least one PPG signal to thereby indicating said inflammatory status of said patient.

It is another object of the present invention to provide the method as defined above, wherein at least one step selected from said receiving at least one PPG signal, analyzing said at least one PPG signal and any combination thereof is continuously measured.

It is another object of the present invention to provide the method as defined above, additionally comprising step of providing at least one baseline pattern of said PPG signal for each of said inflammatory status, such that a deviation above a predetermined threshold from said at least one baseline pattern indicates a change of said inflammatory status.

It is another object of the present invention to provide the method as defined above, additionally comprising step of providing the patient's medical history.

It is another object of the present invention to provide the method as defined above, wherein said at least one PPG signal is obtained by at least one wearable device, a patch placed on said patient's skin, subcutaneous implant, noncontact measurement or any combination thereof.

It is another object of the present invention to provide the method as defined above, wherein said at least one PPG signal is obtained by implantable device.

It is another object of the present invention to provide the method as defined above, wherein said step of analysis is performed by at least one artificial intelligence modality, machine-learning (ML) modality, neural network, deep learning, artificial neural network and any combination thereof.

It is another object of the present invention to provide the method as defined above, wherein said step of analysis additionally comprising step of extracting from said at least one PPG signal over time at least one feature; said at least one feature is selected from a group consisting of time difference between a first peak and a subsequent peak, RR, time difference between a first trough and a subsequent trough, PI, time difference between the first peak and the dicrotic notch, DIT, ratio DIT/RR, normalized ratio DIT/RR, BoA feature, MSL feature, low frequency sum, LF, high frequency sum, HF, DC feature, AC feature, peripheral index feature, ratio peripheral indices and any combination thereof.

It is another object of the present invention to provide the method as defined above, wherein said inflammatory status is selected from a group consisting of: a trajectory of inflammatory flare-up, a trajectory of inflammatory remission, a failure of anti-inflammatory treatment and any combination thereof.

It is another object of the present invention to provide the method as defined above, further comprising step of producing at least one notification pertaining to said inflammatory status.

It is another object of the present invention to provide the method as defined above, wherein said notification includes information selected from a group consisting of said inflammatory status, suggested treatment, a change of treatment corresponding to said inflammatory status and any combination thereof.

It is another object of the present invention to provide the method as defined above, wherein said suggested treatment includes at least one selected from a group consisting of administration of at least one pharmacological agent.

It is another object of the present invention to provide the method as defined above, wherein said at least one pharmacological agent is selected from a group consisting of anti-inflammatory drugs, steroids, immunosuppressives, anti-inflammatory monoclonal antibodies and, anti-inflammatory molecules any combination thereof.

It is another object of the present invention to provide the method as defined above, wherein said inflammatory status pertains to at least one disease selected from a group consisting of immune mediated diseases; said immune mediated diseases being selected from a group consisting of Inflammatory Bowel Diseases (IBD) including ulcerative colitis (UC) and Crohn's disease (CD), Rheumatoid Arthritis (RA), Psoriatic Arthritis (PSA), Spondyloarthritis (SpA), Psoriasis and Chronic Obstructive Pulmonary Disease (COPD), Asthma, Systemic Lupus Erythematosus (SLE), Multiple Sclerosis (MS), Malignant diseases, Cardiovascular diseases, Immune-mediated heart diseases, Vasculitis, Infection-Related Immune-Mediated Diseases and any combination thereof.

It is another object of the present invention to provide the method as defined above, wherein said at least one PPG signal is obtained by a device comprising at least one fixation element adapted to apply pressure on said device such that said device is stabilized and maintained pressed against a predetermined location on the patient's skin, from which said at least one PPG signal is obtained.

It is another object of the present invention to provide the method as defined above, wherein said predetermined location on the patient's skin is selected from a group consisting of at least one finger, arm, forearm, wrist, ear, leg, ankle, scalp, abdominal, thoracic areas and any combination thereof.

It is another object of the present invention to provide the method as defined above, wherein said fixation element is selected from a group consisting of at least one electro-mechanical element, at least one inflating balloon, a spring-based mechanism, a shape-memory alloy-based mechanism and any combination thereof.

It is another object of the present invention to provide the method as defined above, wherein said fixation element is adapted to apply pressure on said device such that said device is stabilized and maintained pressed against a predetermined location on the patient's skin, from which said at least one PPG signal is obtained, in a cyclic manner.

It is another object of the present invention to provide the method as defined above, wherein said cyclic manner comprising steps of:
a. immediately before said step of receiving at least one PPG signal, applying pressure and pressing said device against said predetermined location on the patient's skin by said fixation element;
b. immediately after said step of receiving at least one PPG signal, releasing said pressure applied by said fixation element onto said device.

It is another object of the present invention to provide the method as defined above, additionally comprising step of receiving at least one biomarker level pertaining to said patient.

It is another object of the present invention to provide the method as defined above, wherein said step of receiving at least one biomarker level pertaining to said patient is provided by measurement selected from a group consisting of spectroscopy analysis, non-invasive optical measurements and any combination thereof of samples selected from a group consisting of saliva, blood, urine and any combination thereof.

It is another object of the present invention to provide the method as defined above, wherein said biomarker data includes at least one selected from a group consisting of value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value, d-dimer value blood transcriptomics, proteomics, metabolomics, microbiome and any combination thereof.

It is another object of the present invention to provide the method as defined above, additionally comprising step of obtaining at least one haemorheology parameter associated with blood flow haemorheology.

It is another object of the present invention to provide the method as defined above, wherein said at least one blood flow haemorheology parameter is selected from a group consisting of pulse wave velocity, physiological parameters, digital biomarkers and any combination thereof, wherein digital biomarkers is selected from a group consisting of sleep patterns, steps count, indoor patterns, outdoor patterns, movement patterns, geolocation information, accelerometry information, actigraphy information, mobile use information and any combination thereof.

It is another object of the present invention to provide the method as defined above, additionally comprising step of obtaining at least one blood flow rheological parameter associated with blood flow rheological properties.

It is another object of the present invention to provide the method as defined above, wherein said at least one blood flow rheological parameter is adapted to indicate at least one selected from a group consisting of erythrocytes aggregability and deformability, blood flow, plasma viscosity, vascular resistance, hematocrit and any combination thereof.

It is another object of the present invention to provide the method as defined above, wherein said at least one blood flow rheological parameter indicates said inflammatory status.

It is another object of the present invention to provide the method as defined above, additionally comprising step of providing at least one baseline pattern of at least one blood flow rheological parameter for each of said inflammatory status, such that a deviation above a predetermined threshold from said at least one baseline pattern indicates a change of said inflammatory status.

It is another object of the present invention to provide the method as defined above, additionally comprising steps of
a. momentarily, for a predetermined period of time, reducing blood flow from reaching said at least one location pertaining to said patient; thereby said at least one PPG signal is attenuated;
b. after said predetermined period of time, enabling said blood flow to reach said at least one location pertaining to said patient; thereby said at least one PPG signal is regained;
c. analyzing at least one feature of the attenuated signal versus the regained signal;
   wherein said inflammatory status is provided based on said analysis.

It is another object of the present invention to provide the method as defined above, wherein said step of analyzing additionally comprising step of measuring the amount of time needed for said at least one PPG signal to regain; wherein said inflammatory status is provided based on said amount of time needed for said at least one PPG signal to regain.

It is another object of the present invention to provide the method as defined above, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

It is another object of the present invention to provide the method as defined above, wherein said step of momentarily reducing blood flow is performed by applying pressure on at least one predetermined location.

It is another object of the present invention to provide the method as defined above, wherein said step of momentarily reducing blood flow is performed by means of at least one selected from a group consisting of a cuff, an inflatable cuff, at least one electro-mechanical element, at least one inflating balloon, a spring-based mechanism, a shape-memory alloy-based mechanism and any combination thereof, at least partially encircling said at least one predetermined location.

It is another object of the present invention to provide the method as defined above, additionally comprising steps of
a. applying, for a predetermined period of time, at least one vibration on said at least one location; thereby said at least one PPG signal is changed;
b. after said predetermined period of time, removing said applied vibration; thereby said at least one PPG signal is regained;
c. analyzing at least one feature of at least one selected from a group consisting of the changed signal, the changed signal versus the regained signal and any combination thereof;
   wherein said inflammatory status is provided based on said analysis.

It is another object of the present invention to provide the method as defined above, wherein said feature is selected from a group consisting of the amount of time needed for said at least one PPG signal to regain; the advancement rate of said vibrations from said at least one location along an advancement path, the intensity thereof and any combination thereof.

It is another object of the present invention to provide the method as defined above, wherein said at least one predetermined location is selected from a group consisting of armpit, forearm, finger, leg, ankle, wrist, ear and any combination thereof.

It is another object of the present invention to provide the method as defined above, additionally comprising step of inducing acceleration of blood in said at least one location pertaining to said patient wherein said at least one PPG signal is obtained.

It is another object of the present invention to provide the method as defined above, wherein said step of inducing acceleration of blood in said at least one location is performed by applying shear force on the same.

It is another object of the present invention to provide the method as defined above, wherein said applying shear force is performed by maneuvering said at least one location pertaining to the patient at at least one selected from a group consisting of predetermined speed, predetermined rhythm, for a predetermined period of time and any combination thereof.

It is another object of the present invention to provide the method as defined above, additionally comprising step of analyzing changes in said at least one PPG signal before and after said step of applying shear force on blood in said at least one location pertaining to said patient thereby indicating said inflammatory status of said patient.

It is another object of the present invention to provide the method as defined above, wherein said step of applying shear force is measured by communicating at least one accelerometer with said at least one location pertaining to said patient.

It is another object of the present invention to provide the method as defined above, wherein said step of applying shear force is performed after a request is made to said patient to move said at least one location.

It is another object of the present invention to provide the method as defined above, additionally comprising steps of
a. inducing acceleration of blood in said at least one location is performed by applying shear force on the same; thereby elevating the PPG signal;
b. measuring the amount of time needed for said at least one PPG signal to regain;
   wherein said inflammatory status is provided based on said amount of time needed for said at least one PPG signal to regain.

It is another object of the present invention to provide the method as defined above, wherein said amount of time needed for said at least one PPG signal to regain is indicative of at least one blood flow rheological parameter selected from a group consisting of erythrocytes aggregability and deformability, blood flow, plasma viscosity, vascular resistance, hematocrit and any combination thereof.

It is another object of the present invention to provide the method as defined above, wherein said applying shear force is performed by maneuvering said at least one location pertaining to the patient at at least one selected from a group consisting of predetermined speed, predetermined rhythm, for a predetermined period of time and any combination thereof.

It is another object of the present invention to provide the method as defined above, additionally comprising step of receiving at least one signal, by at least one sensor, pertaining to movement of said patient.

It is another object of the present invention to provide the method as defined above, wherein said at least one sensor is selected from a group consisting of accelerometer, a camera, a microphone, a step counter, a sleep quality sensor and any combination thereof.

It is another object of the present invention to provide the method as defined above, additionally comprising step of receiving at least one signal, by at least one sensor, selected from a group consisting of ambient light sensor, a thermometer, and any combination thereof.

It is another object of the present invention to provide the method as defined above, additionally comprising step of measuring changes in pulse wave velocity, PWV.

It is another object of the present invention to provide the method as defined above, wherein said changes in said PWV are indicative of arterial stiffness.

It is another object of the present invention to provide the method as defined above, wherein increase in said arterial stiffness is indicative of inflammation and reduction in said arterial stiffness is indicative of inflammatory remission.

It is another object of the present invention to provide the method as defined above, wherein said step of measuring changes in said PWV is performed by calculating the pulse transit time, PTT, between at least two pulse waves propagating on the same cardiac cycle from two arterial sites.

It is another object of the present invention to provide the method as defined above, wherein PWV=√(E×h/2rp), where: E=Young's modulus of elasticity of wall material; h=wall thickness of vessel; r-inside radius of vessel; and p-density of blood.

It is another object of the present invention to provide the method as defined above, wherein said at least two pulse waves propagating on the same cardiac cycle from two arterial sites are selected from a group consisting of a pair of PPG signals, a pair of PPG and Electrocardiogram (ECG) signals.

It is another object of the present invention to provide the method as defined above, additionally comprising step of receiving behaviorome data pertaining to said patient.

It is another object of the present invention to provide the method as defined above, wherein said behaviorome data is obtained from sensors selected from a group consisting of indoor and outdoor step counts, motion sensors, geolocators, sleep patterns; indoor and outdoor duration patterns; eating habits/appetite patterns; number of daily visits in the restrooms, motion sensors, geolocators, UV sensors, heart rate sensor, daily steps counter patterns, body temperature sensors, humidity sensor, ambient light sensor, movement patterns, geolocation information, accelerometry information, actigraphy information, mobile use information and any combination thereof.

It is another object of the present invention to provide the method as defined above, additionally comprising step of analyzing said at least one PPG signal to thereby provide a prediction of the future inflammatory status of said patient.

It is another object of the present invention to provide a system for indicating inflammatory status in a patient, comprising:
a monitoring device, adapted to obtain at least one PPG signal from at least one location pertaining to said patient;
a processor in communication with said monitoring device, adapted to analyze said at least one PPG signal to thereby indicate said inflammatory status of said patient.

It is another object of the present invention to provide the system as defined above, wherein said processor is either in direct or indirect physical communication with said monitoring device.

It is another object of the present invention to provide the system as defined above, wherein said processor is in wirelessly communication with said monitoring device.

It is another object of the present invention to provide the system as defined above, wherein said monitoring device is adapted to continuously provide said at least one PPG signal.

It is another object of the present invention to provide the system as defined above, wherein said processor is adapted to provide at least one baseline pattern of said PPG signal for each of said inflammatory status, such that a deviation above a predetermined threshold from said at least one baseline pattern indicates a change of said inflammatory status.

It is another object of the present invention to provide the system as defined above, wherein said processor performs said analysis by at least one artificial intelligence modality, machine-learning (ML) modality, neural network, deep learning, artificial neural network and any combination thereof.

It is another object of the present invention to provide the system as defined above, wherein said processor is adapted to extract from said PPG signal over time at least one feature; said at least one feature is selected from a group consisting of time difference between a first peak and a subsequent peak, RR, time difference between a first trough and a subsequent trough, PI, time difference between the first peak and the dicrotic notch, DIT, ratio DIT/RR, normalized ratio DIT/RR, BoA feature, MSL feature, low frequency sum, LF, high frequency sum, HF, DC feature, AC feature, peripheral index feature, ratio of peripheral indices and any combination thereof.

It is another object of the present invention to provide the system as defined above, wherein said inflammatory status is selected from a group consisting of: a trajectory of inflammatory flare-up, a trajectory of inflammatory remission, a failure of anti-inflammatory treatment and any combination thereof.

It is another object of the present invention to provide the system as defined above, additionally comprising at least one notification system adapted to provide notification pertaining to said inflammatory status.

It is another object of the present invention to provide the system as defined above, wherein said notification includes information selected from a group consisting of said inflammatory status, suggested treatment, a change of treatment corresponding to said inflammatory status and any combination thereof.

It is another object of the present invention to provide the system as defined above, wherein said suggested treatment includes at least one selected from a group consisting of administration of at least one pharmacological agent.

It is another object of the present invention to provide the system as defined above, wherein said at least one pharmacological agent is selected from a group consisting of anti-inflammatory drugs, steroids, immunosuppressives, anti-inflammatory monoclonal antibodies, anti-inflammatory molecules and any combination thereof.

It is another object of the present invention to provide the system as defined above, wherein said inflammatory status pertains to at least one disease selected from a group consisting of immune mediated diseases; said immune mediated diseases being selected from a group consisting of Inflammatory Bowel Diseases (IBD) including ulcerative colitis (UC) and Crohn's disease (CD), Rheumatological diseases, Rheumatoid Arthritis (RA), Psoriatic Arthritis (PsA), Spondyloarthritis (SpA), Psoriasis and Chronic Obstructive Pulmonary Disease (COPD), Asthma, Systemic Lupus Erythematosus (SLE), Multiple Sclerosis (MS), malignant diseases, Vasculitis, cardiovascular diseases, Immune-Mediated Heart Diseases, Infection-Related Immune-Mediated Diseases and any combination thereof.

It is another object of the present invention to provide the system as defined above, wherein said monitoring device additionally comprising fixation element adapted to apply pressure on said monitoring device such that said monitoring device is stabilized and maintained pressed against a predetermined location on the patient's skin, from which said PPG signal is obtained.

It is another object of the present invention to provide the system as defined above, wherein said predetermined location on the patient's skin is selected from a group consisting of at least one finger, arm, forearm, wrist, ear, leg, ankle, scalp, abdominal, thoracic areas and any combination thereof.

It is another object of the present invention to provide the system as defined above, wherein said fixation element is selected from a group consisting of at least one electro-mechanical element, at least one inflating balloon, a spring-based mechanism, a shape-memory alloy-based mechanism and any combination thereof.

It is another object of the present invention to provide the system as defined above, wherein said fixation element is adapted to apply pressure on said monitoring device such that said monitoring device is stabilized and maintained pressed against a predetermined location on the patient's skin, from which said PPG signal is obtained, in a cyclic manner.

It is another object of the present invention to provide the system as defined above, wherein said cyclic manner comprising steps of:
a. immediately before said step of receiving at least one PPG signal, applying pressure and pressing said device against said predetermined location on the patient's skin by said fixation element;
b. immediately after said step of receiving at least one PPG signal, releasing said pressure applied by said fixation element onto said device.

It is another object of the present invention to provide the system as defined above, wherein said monitoring device additionally comprising means of receiving at least one biomarker level pertaining to said patient.

It is another object of the present invention to provide the system as defined above, wherein said means of receiving at least one biomarker level pertaining to said patient is selected from a group consisting of measurement selected from a group consisting of spectroscopy analysis, non-invasive optical measurements and any combination thereof of samples selected from a group consisting of saliva, blood, urine and any combination thereof.

It is another object of the present invention to provide the system as defined above, wherein said biomarker data includes at least one selected from a group consisting of value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value, d-dimer value, blood transcriptomics, proteomics, metabolomics, microbiome and any combination thereof.

It is another object of the present invention to provide the system as defined above, wherein said monitoring device additionally comprising means of obtaining at least one haemorheology parameter associated with blood flow haemorheology.

It is another object of the present invention to provide the system as defined above, wherein said at least one blood flow haemorheology parameter is selected from a group consisting of pulse wave velocity, physiological parameters, digital biomarkers and any combination thereof, wherein digital biomarkers is selected from a group consisting of sleep patterns, steps count, indoor patterns, outdoor patterns, movement patterns, geolocation information, accelerometry information, actigraphy information, mobile use information and any combination thereof.

It is another object of the present invention to provide the system as defined above, wherein said monitoring device additionally comprising means of obtaining at least one blood flow rheological parameter associated with blood flow rheological properties.

It is another object of the present invention to provide the system as defined above, wherein said at least one blood flow rheological parameter is adapted to indicate at least one selected from a group consisting of erythrocytes aggregability and deformability, blood flow, plasma viscosity, vascular resistance, hematocrit any combination thereof.

It is another object of the present invention to provide the system as defined above, wherein said at least one blood flow rheological parameter indicates said inflammatory status.

It is another object of the present invention to provide the system as defined above, wherein said processor is adapted to provide at least one baseline pattern of at least one blood flow rheological parameter for each of said inflammatory status, such that a deviation above a predetermined threshold from said at least one baseline pattern indicates a change of said inflammatory status.

It is another object of the present invention to provide the system as defined above, additionally comprising means adapted to
a. momentarily, for a predetermined period of time, reduce blood flow from reaching said at least one location pertaining to said patient; thereby said at least one PPG signal is attenuated;
b. after said predetermined period of time, enable said blood flow to reach said at least one location pertaining to said patient; thereby said at least one PPG signal is regained;

It is another object of the present invention to provide the system as defined above, wherein said processor is adapted to analyze at least one feature of the attenuated signal versus the regained signal; wherein said inflammatory status is provided based on said analysis.

It is another object of the present invention to provide the system as defined above, wherein said processor is adapted to measure the amount of time needed for said at least one PPG signal to regain; wherein said inflammatory status is provided based on said amount of time needed for said at least one PPG signal to regain.

It is another object of the present invention to provide the system as defined above, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

It is another object of the present invention to provide the system as defined above, wherein said means of momentarily reducing blood flow is performed by applying pressure on at least one predetermined location.

It is another object of the present invention to provide the system as defined above, wherein said step of momentarily reducing blood flow is performed by means of at least one selected from a group consisting of a cuff, an inflatable cuff, at least one electro-mechanical element, at least one inflating balloon, a spring-based mechanism, a shape-memory alloy-based mechanism and any combination thereof, at least partially encircling said at least one predetermined location.

It is another object of the present invention to provide the system as defined above, additionally comprising means adapted to
a. apply, for a predetermined period of time, at least one vibration on said at least one location; thereby said at least one PPG signal is changed;
b. after said predetermined period of time, remove said applied vibration; thereby said at least one PPG signal is regained;
c. analyze at least one feature of at least one selected from a group consisting of the changed signal, the changed signal versus the regained signal and any combination thereof;
   wherein said inflammatory status is provided based on said analysis.

It is another object of the present invention to provide the system as defined above, wherein said feature is selected from a group consisting of the amount of time needed for said at least one PPG signal to regain; the advancement rate of said vibrations from said at least one location along an advancement path, the intensity thereof and any combination thereof.

It is another object of the present invention to provide the system as defined above, wherein said at least one predetermined location is selected from a group consisting of armpit, forearm, wrist, ear, leg, ankle, scalp, abdominal and thoracic areas and any combination thereof.

It is another object of the present invention to provide the system as defined above, additionally comprising means of inducing acceleration of blood in said at least one location pertaining to said patient where said at least one PPG signal is received.

It is another object of the present invention to provide the system as defined above, wherein said means of inducing acceleration of blood in said at least one location is performed by applying shear force on the same.

It is another object of the present invention to provide the system as defined above, wherein said processor is adapted to analyze changes in said at least one PPG signal before and after said applying shear force on blood in said at least one location pertaining to said patient; and, thereby to indicate said inflammatory status of said patient.

It is another object of the present invention to provide the system as defined above, additionally comprising means adapted to induce acceleration of blood in said at least one location is performed by applying shear force on the same; thereby elevating the PPG signal.

It is another object of the present invention to provide the system as defined above, wherein said applying shear force is performed by maneuvering said at least one location pertaining to the patient at at least one selected from a group consisting of predetermined speed, predetermined rhythm, for a predetermined period of time and any combination thereof.

It is another object of the present invention to provide the system as defined above, wherein said processor is adapted to analyze at least one feature of the attenuated signal versus the regained signal; wherein said inflammatory status is provided based on said analysis.

It is another object of the present invention to provide the system as defined above, wherein said processor is adapted to measure the amount of time needed for said at least one PPG signal to regain; wherein said inflammatory status is provided based on said amount of time needed for said at least one PPG signal to regain.

It is another object of the present invention to provide the system as defined above, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

It is another object of the present invention to provide a method of indicating at least one inflammatory status of a patient, comprising steps of:
(a) illuminating at least one location pertaining to the patient with at least one optical light beam characterized by at least one wavelength;
(b) detecting at least one signal; said signal is at least a portion of at least one selected from a group consisting of the transmitted light beam, the absorbed light beam, the reflected light beam and any combination thereof; and,
(c) analyzing said at least a portion of said signal as a function of time thereby indicating said inflammatory status of said patient;
wherein said step of analyzing at least a portion of said signal additionally comprising step of: (a) extracting at least one feature; (b) analyzing at least one trend of said at least one feature as a function of time; and, (c) correlating said at least one trend with at least one biomarker indicative of said at least one inflammatory status.

It is another object of the present invention to provide the method as defined above, wherein at least one step selected from a group consisting of illuminating, detecting, analyzing and any combination thereof is performed continuously.

It is another object of the present invention to provide the method as defined above, wherein said step of detecting is performed by at least one sensor selected from a group consisting of photodiode, laser light source and any combination thereof.

It is another object of the present invention to provide the method as defined above, wherein said step of illuminating at least one optical light beam is performed in a manner selected from pulsed, continues and any combination thereof.

It is another object of the present invention to provide the method as defined above, wherein said at least one wavelength is in the range of about 200 nm to about 800 nm and/or 1 mm to 700 nm.

It is another object of the present invention to provide the method as defined above, wherein said steps of (a) illuminating at least one location pertaining to the patient with at least one optical light beam characterized by at least one wavelength; and step of (b) detecting at least a portion of at least one selected from a group consisting of the transmitted light beam, the absorbed light beam, the reflected light beam and any combination thereof; is performed by at least one photoplethysmogram (PPG).

It is another object of the present invention to provide the method as defined above, wherein said step of analyzing said at least a portion of said signal is performed by analyzing at least one photoplethysmography (PPG) signal from at least one location pertaining to the patient by means of said at least one photoplethysmogram (PPG).

It is another object of the present invention to provide the method as defined above, wherein said step of illuminating at least one location pertaining to the patient with at least one optical light beam characterized by at least one wavelength is performed by at least one optical source selected from a group consisting of photodiode, laser light source and any combination thereof.

It is another object of the present invention to provide the method as defined above, wherein said step of analyzing said at least a portion of said signal as a function of time, additionally comprising step of analyzing the intensity of said signal as a function of time.

It is another object of the present invention to provide the method as defined above, wherein said step of analyzing said at least a portion said signal as a function of time, additionally comprising step of providing at least one reference signal as a function of time.

It is another object of the present invention to provide the method as defined above, wherein said at least one reference signal is at least one selected from a group consisting of the transmitted light beam, the absorbed light beam, the reflected light beam and any combination thereof as a function of time, of at least one selected from a group consisting of said patient with a known inflammatory status, of at least one group of patients of with known inflammatory status, an average of at least one group of patients of with known inflammatory status, and any combination thereof.

It is another object of the present invention to provide the method as defined above, wherein said known inflammatory status is selected from a group consisting of a flare-up, a remission, a healthy pattern and any combination thereof.

It is another object of the present invention to provide the method as defined above, wherein said step of analyzing said at least a portion of said signal as a function of time, additionally comprising step of comparing and identifying deviations of the same with said at least one reference signal as a function of time.

It is another object of the present invention to provide the method as defined above, additionally comprising step of providing at least one baseline pattern of said PPG signal for each of said inflammatory status, such that a deviation above a predetermined threshold from said at least one baseline pattern indicates a change of said inflammatory status.

It is another object of the present invention to provide the method as defined above, wherein said baseline pattern represents at least one selected from a group consisting if a flare-up, a remission, a healthy pattern and any combination thereof.

It is another object of the present invention to provide the method as defined above, additionally comprising step of providing the patient's medical history.

It is another object of the present invention to provide the method as defined above, wherein said at least one PPG signal is obtained by at least one wearable device, a patch placed on said patient's skin, noncontact measurement or any combination thereof.

It is another object of the present invention to provide the method as defined above, wherein said at least one signal is obtained by an implantable device.

It is another object of the present invention to provide the method as defined above, wherein said step of analysis is performed by at least one artificial intelligence modality, machine-learning (ML) modality, neural network, deep learning, artificial neural network and any combination thereof.

It is another object of the present invention to provide the method as defined above, wherein said at least one feature is selected from a group consisting of time difference between a first peak and a subsequent peak, RR, time difference between a first trough and a subsequent trough, PI, time difference between the first peak and the dicrotic notch, DIT, ratio DIT/RR, normalized ratio DIT/RR, BoA feature, MSL feature, low frequency sum, LF, high frequency sum, HF, DC feature, AC feature, peripheral index feature, ratio of peripheral indices and any combination thereof.

It is another object of the present invention to provide the method as defined above, wherein said inflammatory status is selected from a group consisting of: a trajectory of inflammatory flare-up, a trajectory of inflammatory remission, a failure of anti-inflammatory treatment and any combination thereof.

It is another object of the present invention to provide the method as defined above, further comprising step of producing at least one notification pertaining to said inflammatory status.

It is another object of the present invention to provide the method as defined above, wherein said notification includes information selected from a group consisting of said inflammatory status, suggested treatment, a change of treatment corresponding to said inflammatory status and any combination thereof.

It is another object of the present invention to provide the method as defined above, wherein said suggested treatment includes at least one selected from a group consisting of administration of at least one pharmacological agent.

It is another object of the present invention to provide the method as defined above, wherein said at least one pharmacological agent is selected from a group consisting of anti-inflammatory drugs, steroids, immunosuppressives, anti-inflammatory monoclonal antibodies, anti-inflammatory molecules and any combination thereof.

It is another object of the present invention to provide the method as defined above, wherein said inflammatory status pertains to at least one disease selected from a group consisting of immune mediated diseases; said immune mediated diseases being selected from a group consisting of Inflammatory Bowel Diseases (IBD) including ulcerative colitis (UC) and Crohn's disease (CD), Rheumatological diseases, Rheumatoid Arthritis (RA), Psoriatic Arthritis (PsA), Spondyloarthritis (SpA) Psoriasis, Chronic Obstructive Pulmonary disease (COPD), Asthma, Systemic Lupus Erythematosus (SLE), Multiple Sclerosis (MS), Immune-mediated heart diseases, Vasculitis, malignant diseases, cardiovascular diseases, Immune-Mediated Heart Diseases, Infection-Related Immune-Mediated Diseases, and any combination thereof.

It is another object of the present invention to provide the method as defined above, wherein said at least one PPG signal is obtained by a device comprising fixation element adapted to apply pressure on said device such that said device is stabilized and maintained pressed against a predetermined location on the patient's skin, from which said at least one PPG signal is obtained.

It is another object of the present invention to provide the method as defined above, wherein said predetermined location on the patient's skin is selected from a group consisting of at least one finger, arm, forearm, wrist, ear, leg, ankle, scalp, abdominal, thoracic areas and any combination thereof.

It is another object of the present invention to provide the method as defined above, wherein said fixation element is selected from a group consisting of at least one electro-mechanical element, at least one inflating balloon, a spring-based mechanism, a shape-memory alloy-based mechanism and any combination thereof.

It is another object of the present invention to provide the method as defined above, wherein said fixation element is adapted to apply pressure on said device such that said device is maintained pressed against a predetermined location on the patient's skin, from which said at least one PPG signal is obtained, in a cyclic manner.

It is another object of the present invention to provide the method as defined above, wherein said cyclic manner comprising steps of:
a. immediately before said step of receiving at least one PPG signal, applying pressure and pressing said device against said predetermined location on the patient's skin by said fixation element;
b. immediately after said step of receiving at least one PPG signal, releasing said pressure applied by said fixation element onto said device.

It is another object of the present invention to provide the method as defined above, additionally comprising step of receiving at least one biomarker level pertaining to said patient.

It is another object of the present invention to provide the method as defined above, wherein said step of receiving at least one biomarker level pertaining to said patient is provided by measurement selected from a group consisting of spectroscopy analysis, non-invasive optical measurements and any combination thereof of samples selected from a group consisting of saliva, blood, urine and any combination thereof.

It is another object of the present invention to provide the method as defined above, wherein said biomarker data includes at least one selected from a group consisting of value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value, d-dimer value and blood transcriptomics, proteomics, metabolomics, microbiome and combination thereof.

It is another object of the present invention to provide the method as defined above, additionally comprising step of obtaining at least one haemorheology parameter associated with blood flow haemorheology.

It is another object of the present invention to provide the method as defined above, wherein said at least one blood flow haemorheology parameter is selected from a group consisting of pulse wave velocity, physiological parameters, digital biomarkers and any combination thereof, wherein digital biomarkers is selected from a group consisting of sleep patterns, steps count, indoor patterns, outdoor patterns, movement patterns, geolocation information, accelerometry information, actigraphy information, mobile use information and any combination thereof.

It is another object of the present invention to provide the method as defined above, additionally comprising step of obtaining at least one blood flow rheological parameter associated with blood flow rheological properties.

It is another object of the present invention to provide the method as defined above, wherein said at least one blood flow rheological parameter is adapted to indicate at least one selected from a group consisting of erythrocytes aggregability and deformability, blood flow, plasma viscosity, vascular resistance, hematocrit and any combination thereof.

It is another object of the present invention to provide the method as defined above, wherein said at least one blood flow rheological parameter indicates said inflammatory status.

It is another object of the present invention to provide the method as defined above, additionally comprising step of providing at least one baseline pattern of at least one blood flow rheological parameter for each of said inflammatory status, such that a deviation above a predetermined threshold from said at least one baseline pattern indicates a change of said inflammatory status.

It is another object of the present invention to provide the method as defined above, additionally comprising steps of
a. momentarily, for a predetermined period of time, reducing blood flow from reaching said at least one location pertaining to said patient; thereby said at least one PPG signal is attenuated;
b. after said predetermined period of time, enabling said blood flow to reach said at least one location pertaining to said patient; thereby said at least one PPG signal is regained;
c. analyzing at least one feature of the attenuated signal versus the regained signal;
   wherein said inflammatory status is provided based on said analysis.

It is another object of the present invention to provide the method as defined above, wherein said step of analyzing additionally comprising step of measuring the amount of time needed for said at least one PPG signal to regain; wherein said inflammatory status is provided based on said amount of time needed for said at least one PPG signal to regain.

It is another object of the present invention to provide the method as defined above, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

It is another object of the present invention to provide the method as defined above, wherein said step of momentarily reducing blood flow is performed by applying pressure on at least one predetermined location.

It is another object of the present invention to provide the method as defined above, wherein said step of momentarily reducing blood flow is performed by means of at least one selected from a group consisting of a cuff, an inflatable cuff, at least one electro-mechanical element, at least one inflating balloon, a spring-based mechanism, a shape-memory alloy-based mechanism and any combination thereof, at least partially encircling said at least one predetermined location.

It is another object of the present invention to provide the method as defined above, wherein said steps of (a) momentarily reducing blood flow; and, said step of (b) enabling said blood flow to reach said at least one location pertaining to said patient, are synchronized with said step of detecting at least one signal; such that said signal is synchronized and detected immediately after said step of momentarily reducing blood flow; and immediately after said step of enabling said blood flow to reach said at least one location pertaining to said patient.

It is another object of the present invention to provide the method as defined above, additionally comprising steps of:
a. applying, for a predetermined period of time, at least one vibration on said at least second location, substantially different from said at least one location; thereby said at least one signal is changed;
b. after said predetermined period of time, removing said applied vibration; thereby said at least one signal is regained;
c. analyzing at least one feature of at least one selected from a group consisting of the changed signal, the changed signal versus the regained signal and any combination thereof;
   wherein said inflammatory status is provided based on said analysis.

It is another object of the present invention to provide the method as defined above, wherein said feature is selected from a group consisting of the amount of time needed for said at least one signal to regain; the advancement rate of said vibrations from said at least one second location to said at least one location, the intensity thereof as a function of time, and any combination thereof.

It is another object of the present invention to provide the method as defined above, wherein said at least one predetermined location is selected from a group consisting of armpit, forearm, finger, leg, ankle, wrist, ear and any combination thereof.

It is another object of the present invention to provide the method as defined above, additionally comprising step of inducing acceleration of blood in said at least one location pertaining to said patient where said at least one signal is received.

It is another object of the present invention to provide the method as defined above, wherein said step of inducing acceleration of blood in said at least one location is performed by applying shear force on the same.

It is another object of the present invention to provide the method as defined above, additionally comprising step of analyzing changes in said at least one signal before and after said step of applying shear force on blood in said at least one location pertaining to said patient thereby indicating said inflammatory status of said patient.

It is another object of the present invention to provide the method as defined above, wherein said step of applying shear force is measured by communicating at least one accelerometer with said at least one location pertaining to said patient.

It is another object of the present invention to provide the method as defined above, wherein said applying shear force is performed by maneuvering said at least one location pertaining to the patient at at least one selected from a group consisting of predetermined speed, predetermined rhythm, for a predetermined period of time and any combination thereof.

It is another object of the present invention to provide the method as defined above, additionally comprising steps of
a. inducing acceleration of blood in said at least one location is performed by applying shear force on the same; thereby elevating the signal;
b. analyzing at least one feature of the attenuated signal versus the regained signal;
wherein said inflammatory status is provided based on said analysis.

It is another object of the present invention to provide the method as defined above, wherein said step of analyzing additionally comprising step of measuring the amount of time needed for said at least one signal to regain; wherein said inflammatory status is provided based on said amount of time needed for said at least one signal to regain.

It is another object of the present invention to provide the method as defined above, wherein said feature is selected from a group consisting of the amount of time needed for said at least one signal to regain, the rate at which said at least one signal is regained, the intensity of said attenuated signal, the integral of the signal as a function of time, the derivative of the signal as a function of time and any combination thereof.

It is another object of the present invention to provide the method as defined above, wherein said amount of time needed for said at least one signal to regain is indicative of at least one blood flow rheological parameter selected from a group consisting of erythrocytes aggregability and deformability, blood flow, plasma viscosity, vascular resistance, hematocrit and any combination thereof.

It is another object of the present invention to provide the method as defined above, additionally comprising step of receiving at least one signal, by at least one sensor, pertaining to movement of said patient.

It is another object of the present invention to provide the method as defined above, wherein said at least one sensor is selected from a group consisting of accelerometer, a camera, a microphone, a step counter, a sleep quality sensor and any combination thereof.

It is another object of the present invention to provide the method as defined above, additionally comprising step of receiving at least one signal, by at least one sensor, selected from a group consisting of ambient light sensor, a thermometer, and any combination thereof.

It is another object of the present invention to provide the method as defined above, additionally comprising step of measuring changes in pulse wave velocity, PWV.

It is another object of the present invention to provide the method as defined above, wherein said changes in said PWV are indicative of arterial stiffness.

It is another object of the present invention to provide the method as defined above, wherein increase in said arterial stiffness is indicative of inflammation and reduction in said arterial stiffness is indicative of inflammatory remission.

It is another object of the present invention to provide the method as defined above, wherein said step of measuring changes in said PWV is performed by calculating the pulse transit time, PTT, between at least two pulse waves propagating on the same cardiac cycle from two arterial sites.

It is another object of the present invention to provide the method as defined above, wherein PWV=√(E×h/2rp), where: E=Young's modulus of elasticity of wall material; h-wall thickness of vessel; r-inside radius of vessel; and p-density of blood.

It is another object of the present invention to provide the method as defined above, wherein said at least two pulse waves propagating on the same cardiac cycle from two arterial sites are selected from a group consisting of a pair of PPG signals, a pair of PPG and Electrocardiogram (ECG) signals.

It is another object of the present invention to provide the method as defined above, additionally comprising step of receiving behaviorome data pertaining to said patient.

It is another object of the present invention to provide the method as defined above, wherein said behaviorome data is obtained from sensors selected from a group consisting of indoor and outdoor step counts, motion sensors, geolocators, sleep patterns; indoor and outdoor duration patterns; eating habits/appetite patterns; number of daily visits in the restrooms, motion sensors, geolocators, UV sensors, heart rate sensor, daily steps counter patterns, body temperature sensors, humidity sensor, ambient light sensor, movement patterns, geolocation information, accelerometry information, actigraphy information, mobile use information and any combination thereof.

It is another object of the present invention to provide the method as defined above, additionally comprising step of analyzing said at least one PPG signal to thereby provide a prediction of the future inflammatory status of said patient.

It is another object of the present invention to provide a system for indicating inflammatory status in a patient, comprising:
a monitoring device, adapted to obtain from at least one location pertaining to said patient at least one signal; said signal is at least a portion of at least one selected from a group consisting of a transmitted light beam, an absorbed light beam, a reflected light beam and any combination thereof from at least one optical light beam illuminated on said at least one location;
a processor in communication with said monitoring device, adapted to analyze said at least a portion of at least a portion of at least one selected from a group consisting of the transmitted light beam, the absorbed light beam, the reflected light beam and any combination thereof as a function of time by (a) extracting at least one feature; (b) analyzing at least one trend of said at least one feature as a function of time; and, (c) correlating said at least one trend with at least one biomarker indicative of said at least one inflammatory status, to thereby indicate said inflammatory status of said patient.

It is another object of the present invention to provide the system as defined above, wherein said at least one optical light beam is characterized by at least one wavelength.

It is another object of the present invention to provide the system as defined above, wherein said at least one wavelength is in the range of about 200 nm to about 800 nm and/or 1 mm to 700 nm.

It is another object of the present invention to provide the system as defined above, additionally comprising at least one optical source selected from a group consisting of photodiode, laser light source, and any combination thereof, adapted to illuminate said at least one location pertaining to the patient with at least one optical light beam.

It is another object of the present invention to provide the system as defined above, wherein said at least one optical source is adapted to illuminate said at least one optical light beam in a manner selected from pulsed, continues and any combination thereof.

It is another object of the present invention to provide the system as defined above, additionally comprising at least one photodiode (and/or laser light source) adapted to detect at least a portion of at least a portion of at least one selected from a group consisting of the transmitted light beam, the absorbed light beam, the reflected light beam and any combination thereof as a function of time.

It is another object of the present invention to provide the system as defined above, wherein said monitoring device is at least one photoplethysmogram (PPG).

It is another object of the present invention to provide the system as defined above, wherein processor is adapted to analyze one photoplethysmography (PPG) signal from at least one location pertaining to the patient by means of said at least one photoplethysmogram (PPG).

It is another object of the present invention to provide the system as defined above, wherein processor is adapted to analyze the intensity of at least one selected from a group consisting of the transmitted light beam, the absorbed light beam, the reflected light beam and any combination thereof as a function of time.

It is another object of the present invention to provide the system as defined above, wherein processor is adapted to compare at least a portion of at least one selected from a group consisting of the transmitted light beam, the absorbed light beam, the reflected light beam and any combination thereof as a function of time, with said at least one reference signal as a function of time.

It is another object of the present invention to provide the system as defined above, wherein said at least one reference signal is at least one selected from a group consisting of the transmitted light bean, the absorbed light beam, the reflected light beam and any combination thereof as a function of time, of at least one selected from a group consisting of said patient with a known inflammatory status, at least one group of patients of with known inflammatory status an average of at least one group of patients of with known inflammatory status, and any combination thereof.

It is another object of the present invention to provide the system as defined above, wherein said known inflammatory status is selected from a group consisting of a flare-up, a remission, a healthy pattern and any combination thereof.

It is another object of the present invention to provide the system as defined above, wherein said processor is either in direct or indirect physical communication with said monitoring device.

It is another object of the present invention to provide the system as defined above, wherein said processor is in wirelessly communication with said monitoring device.

It is another object of the present invention to provide the system as defined above, wherein said monitoring device is adapted to continuously provide said at least one PPG signal.

It is another object of the present invention to provide the system as defined above, wherein said processor is adapted to provide at least one baseline pattern of said PPG signal for each of said inflammatory status, such that a deviation above a predetermined threshold from said at least one baseline pattern indicates a change of said inflammatory status.

It is another object of the present invention to provide the system as defined above, wherein said processor performs said analysis by at least one artificial intelligence modality, machine-learning (ML) modality, neural network, deep learning, artificial neural network and any combination thereof.

It is another object of the present invention to provide the system as defined above, wherein said processor is adapted to extract from said PPG signal over time at least one feature; said at least one feature is selected from a group consisting of time difference between a first peak and a subsequent peak, RR, time difference between a first trough and a subsequent trough, PI, time difference between the first peak and the dicrotic notch, DIT, ratio DIT/RR, normalized ratio DIT/RR, BoA feature, MSL feature, low frequency sum, LF, high frequency sum, HF, DC feature, AC feature, peripheral index feature, ratio of peripheral indices and any combination thereof.

It is another object of the present invention to provide the system as defined above, wherein said inflammatory status is selected from a group consisting of: a trajectory of inflammatory flare-up, a trajectory of inflammatory remission, a failure of anti-inflammatory treatment and any combination thereof.

It is another object of the present invention to provide the system as defined above, additionally comprising at least one notification system adapted to provide notification pertaining to said inflammatory status.

It is another object of the present invention to provide the system as defined above, wherein said notification includes information selected from a group consisting of said inflammatory status, suggested treatment, a change of treatment corresponding to said inflammatory status and any combination thereof.

It is another object of the present invention to provide the system as defined above, wherein said suggested treatment includes at least one selected from a group consisting of administration of at least one pharmacological agent.

It is another object of the present invention to provide the system as defined above, wherein said at least one pharmacological agent is selected from a group consisting of anti-inflammatory drugs, steroids, immunosuppressives, anti-inflammatory monoclonal antibodies, anti-inflammatory molecules and any combination thereof.

It is another object of the present invention to provide the system as defined above, wherein said inflammatory status pertains to at least one disease selected from a group consisting of immune mediated diseases; said immune mediated diseases being selected from a group consisting of Inflammatory Bowel Diseases (IBD) including ulcerative colitis (UC) and Crohn's disease (CD), Rheumatological diseases, Rheumatoid Arthritis (RA), Psoriatic Arthritis (PsA), Spondyloarthritis (SpA), Psoriasis, Chronic Obstructive Pulmonary Disease (COPD), Asthma, Systemic Lupus Erythematosus (SLE), Multiple Sclerosis (MS), Vasculitis, malignant diseases, cardiovascular diseases, Immune-Mediated Heart Diseases, Infection-Related Immune-Mediated Diseases and any combination thereof.

It is another object of the present invention to provide the system as defined above, wherein said monitoring device additionally comprising fixation element adapted to apply pressure on said monitoring device such that said monitoring device is stabilized and maintained pressed against a predetermined location on the patient's skin, from which said PPG signal is obtained.

It is another object of the present invention to provide the system as defined above, wherein said predetermined location on the patient's skin is selected from a group consisting of at least one finger, arm, forearm, wrist, ear, leg, ankle, scalp, abdominal, thoracic areas and any combination thereof.

It is another object of the present invention to provide the system as defined above, wherein said fixation element is selected from a group consisting of at least one electro-mechanical element, at least one inflating balloon, a spring-based mechanism, a shape-memory alloy-based mechanism and any combination thereof.

It is another object of the present invention to provide the system as defined above, wherein said fixation element is adapted to apply pressure on said monitoring device such that said monitoring device is stabilized and maintained pressed against a predetermined location on the patient's skin, from which said PPG signal is obtained, in a cyclic manner.

It is another object of the present invention to provide the system as defined above, wherein said cyclic manner comprising steps of:
a. immediately before said step of receiving at least one PPG signal, applying pressure and pressing said device against said predetermined location on the patient's skin by said fixation element;
b. immediately after said step of receiving at least one PPG signal, releasing said pressure applied by said fixation element onto said device.

It is another object of the present invention to provide the system as defined above, wherein said monitoring device additionally comprising means of receiving at least one biomarker level pertaining to said patient.

It is another object of the present invention to provide the system as defined above, wherein said means of receiving at least one biomarker level pertaining to said patient is selected from a group consisting of measurement selected from a group consisting of spectroscopy analysis, non-invasive optical measurements and any combination thereof of samples selected from a group consisting of saliva, blood, urine and any combination thereof.

It is another object of the present invention to provide the system as defined above, wherein said biomarker data includes at least one selected from a group consisting of value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value, d-dimer value blood transcriptomics, proteomics, metabolomics, microbiome and any combination thereof.

It is another object of the present invention to provide the system as defined above, wherein said monitoring device additionally comprising means of receiving at least one haemorheology parameter associated with blood flow haemorheology.

It is another object of the present invention to provide the system as defined above, wherein said at least one blood flow haemorheology parameter is selected from a group consisting of pulse wave velocity, physiological parameters, digital biomarkers and any combination thereof; wherein digital biomarkers is selected from a group consisting of sleep patterns, steps count, indoor patterns, outdoor patterns, movement patterns, geolocation information, accelerometry information, actigraphy information, mobile use information and any combination thereof.

It is another object of the present invention to provide the system as defined above, wherein said monitoring device additionally comprising means of obtaining at least one blood flow rheological parameter associated with blood flow rheological properties.

It is another object of the present invention to provide the system as defined above, wherein said at least one blood flow rheological parameter is adapted to indicate at least one selected from a group consisting of erythrocytes aggregability and deformability, blood flow, plasma viscosity, vascular resistance, hematocrit and any combination thereof.

It is another object of the present invention to provide the system as defined above, wherein said at least one blood flow rheological parameter indicates said inflammatory status.

It is another object of the present invention to provide the system as defined above, wherein said processor is adapted to provide at least one baseline pattern of at least one blood flow rheological parameter for each of said inflammatory status, such that a deviation above a predetermined threshold from said at least one baseline pattern indicates a change of said inflammatory status.

It is another object of the present invention to provide the system as defined above, additionally comprising means adapted to
a. momentarily, for a predetermined period of time, reduce blood flow from reaching said at least one location pertaining to said patient; thereby said at least one PPG signal is attenuated;
b. after said predetermined period of time, enable said blood flow to reach said at least one location pertaining to said patient; thereby said at least one PPG signal is regained;

It is another object of the present invention to provide the system as defined above, wherein said processor is adapted to analyze at least one feature of the attenuated signal versus the regained signal; wherein said inflammatory status is provided based on said analysis.

It is another object of the present invention to provide the system as defined above, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

It is another object of the present invention to provide the system as defined above, wherein said processor is adapted to measure the amount of time needed for said at least one PPG signal to regain; wherein said inflammatory status is provided based on said amount of time needed for said at least one PPG signal to regain.

It is another object of the present invention to provide the system as defined above, wherein said means of momentarily reducing blood flow is performed by applying pressure on at least one predetermined location.

It is another object of the present invention to provide the system as defined above, wherein said step of momentarily reducing blood flow is performed by means of at least one selected from a group consisting of a cuff, an inflatable cuff, at least one electro-mechanical element, at least one inflating balloon, a spring-based mechanism, a shape-memory alloy-based mechanism and any combination thereof, at least partially encircling said at least one predetermined location.

It is another object of the present invention to provide the system as defined above, additionally comprising at least one vibrating element adapted to apply vibration to at least one second location, substantially different from said at least one location.

It is another object of the present invention to provide the system as defined above, additionally comprising means adapted to:
a. apply, for a predetermined period of time, at least one vibration on said at least one second location; thereby said at least one signal is changed;
b. after said predetermined period of time, remove said applied vibration; thereby said at least one signal is regained;
c. analyze at least one feature of at least one selected from a group consisting of the changed signal, the changed signal versus the regained signal and any combination thereof;
   wherein said inflammatory status is provided based on said analysis.

It is another object of the present invention to provide the system as defined above, wherein said feature is selected from a group consisting of the amount of time needed for said at least one signal to regain; the advancement rate of said vibrations from said at least one second location to said at least one location, the intensity thereof as a function of time, and any combination thereof.

It is another object of the present invention to provide the system as defined above, wherein said at least one predetermined location is selected from a group consisting of armpit, forearm, finger, leg, ankle, wrist, ear, and any combination thereof.

It is another object of the present invention to provide the system as defined above, additionally comprising means of inducing acceleration of blood in said at least one location pertaining to said patient where said at least one signal is received.

It is another object of the present invention to provide the system as defined above, wherein said means of inducing acceleration of blood in said at least one location is performed by applying shear force on the same.

It is another object of the present invention to provide the system as defined above, wherein said processor is adapted to analyze changes in said at least one PPG signal before and after said applying shear force on blood in said at least one location pertaining to said patient; and, thereby to indicate said inflammatory status of said patient.

It is another object of the present invention to provide the system as defined above, additionally comprising means adapted to induce acceleration of blood in said at least one location is performed by applying shear force on the same; thereby elevating the PPG signal.

It is another object of the present invention to provide the system as defined above, wherein said applying shear force is performed by maneuvering said at least one location pertaining to the patient at at least one selected from a group consisting of predetermined speed, predetermined rhythm, for a predetermined period of time and any combination thereof.

It is another object of the present invention to provide the system as defined above, wherein said processor is adapted to measure the amount of time needed for said at least one PPG signal to regain; wherein said inflammatory status is provided based on said amount of time needed for said at least one PPG signal to regain.

It is another object of the present invention to provide the system as defined above, wherein said amount of time needed for said at least one PPG signal to regain is indicative of at least one blood flow rheological parameter selected from a group consisting of erythrocytes aggregability and deformability, blood flow, plasma viscosity, vascular resistance, hematocrit and any combination thereof.

It is another object of the present invention to provide the system as defined above, wherein said monitoring device additionally comprising at least one sensor adapted to provide data pertaining to movement of said patient.

It is another object of the present invention to provide the system as defined above, wherein said at least one sensor is selected from a group consisting of accelerometer, a camera, a microphone, a step counter, a sleep quality sensor and any combination thereof.

It is another object of the present invention to provide the system as defined above, wherein said monitoring device additionally comprising at least one sensor selected from a group consisting of ambient light sensor, a thermometer, and any combination thereof.

It is another object of the present invention to provide the system as defined above, wherein said processor adapted to measure changes in pulse wave velocity, PWV.

It is another object of the present invention to provide the system as defined above, wherein said changes in said PWV are indicative of arterial stiffness.

It is another object of the present invention to provide the system as defined above, wherein increase in said arterial stiffness is indicative of inflammation and reduction in said arterial stiffness is indicative of inflammatory remission.

It is another object of the present invention to provide the system as defined above, wherein said changes in said PWV is performed by calculating the pulse transit time, PTT, between at least two pulse waves propagating on the same cardiac cycle from two arterial sites.

It is another object of the present invention to provide the system as defined above, wherein PWV=√(E×h/2rp), where: E=Young's modulus of elasticity of wall material; h-wall thickness of vessel; r=inside radius of vessel; and ρ=density of blood.

It is another object of the present invention to provide the system as defined above, wherein said at least two pulse waves propagating on the same cardiac cycle from two arterial sites are selected from a group consisting of a pair of PPG signals, a pair of PPG and Electrocardiogram (ECG) signals.

It is another object of the present invention to provide the system as defined above, wherein said monitoring device additionally comprising at least one sensor adapted to provide behaviorome data pertaining to said patient.

It is another object of the present invention to provide the system as defined above, wherein said at least one sensor is selected from a group consisting of indoor and outdoor step counts, motion sensors, geolocators, sleep patterns; indoor and outdoor duration patterns; eating habits/appetite patterns; numbers of daily visits in the restrooms, motion sensors, geolocators, UV sensors, heart rate sensor, daily steps counter patterns, body temperature sensors, humidity sensor, ambient light sensor, movement patterns, geolocation information, accelerometry information, actigraphy information, mobile use information and any combination thereof.

It is another object of the present invention to provide the system as defined above, wherein the monitoring device is a non-invasive wearable device.

It is still an object of the present invention to provide the system as defined above, wherein the monitoring device is a patch device.

It is lastly an object of the present invention to provide the system as defined above, wherein the monitoring device is implantable.

It is another object of the present invention to provide the system as defined above, wherein said amount of time needed for said at least one PPG signal to regain is indicative of at least one blood flow rheological parameter selected from a group consisting of erythrocytes aggregability and deformability, blood flow, plasma viscosity, vascular resistance, hematocrit and any combination thereof.

It is another object of the present invention to provide the system as defined above, wherein said monitoring device additionally comprising at least one sensor adapted to provide data pertaining to movement of said patient.

It is another object of the present invention to provide the system as defined above, wherein said at least one sensor is selected from a group consisting of accelerometer, a camera, a microphone, a step counter, a sleep quality sensor and any combination thereof.

It is another object of the present invention to provide the system as defined above, wherein said monitoring device additionally comprising at least one sensor selected from a group consisting of ambient light sensor, a thermometer, and any combination thereof.

It is another object of the present invention to provide the system as defined above, wherein said processor adapted to measure changes in pulse wave velocity, PWV.

It is another object of the present invention to provide the system as defined above, wherein said changes in said PWV are indicative of arterial stiffness.

It is another object of the present invention to provide the system as defined above, wherein increase in said arterial stiffness is indicative of inflammation and reduction in said arterial stiffness is indicative of inflammatory remission.

It is another object of the present invention to provide the system as defined above, wherein said changes in said PWV is performed by calculating the pulse transit time, PTT, between at least two pulse waves propagating on the same cardiac cycle from two arterial sites.

It is another object of the present invention to provide the system as defined above, wherein PWV=√(E×h/2rp), where: E-Young's modulus of elasticity of wall material; h-wall thickness of vessel; r-inside radius of vessel; and ρ=density of blood.

It is another object of the present invention to provide the system as defined above, wherein said at least two pulse waves propagating on the same cardiac cycle from two arterial sites are selected from a group consisting of a pair of PPG signals, a pair of PPG and Electrocardiogram (ECG) signals.

It is another object of the present invention to provide the system as defined above, wherein said monitoring device additionally comprising at least one sensor adapted to provide behaviorome data pertaining to said patient.

It is another object of the present invention to provide the system as defined above, wherein said at least one sensor is selected from a group consisting of indoor and outdoor step counts, motion sensors, geolocators, sleep patterns; indoor and outdoor duration patterns; eating habits/appetite patterns; number of daily visits in the restrooms, motion sensors, geolocators, UV sensors, heart rate sensor, daily steps counter patterns, body temperature sensors, humidity sensor, ambient light sensor, movement patterns, geolocation information, accelerometry information, actigraphy information, mobile use information and any combination thereof.

It is another object of the present invention to provide the system as defined above, wherein the monitoring device is a non-invasive wearable device.

It is still an object of the present invention to provide the system as defined above, wherein the monitoring device is a patch device.

It is lastly an object of the present invention to provide the system as defined above, wherein the monitoring device is implantable.

Embodiments of the invention may include a method of assessment of an inflammatory status in a patient. Embodiments of the method may include receiving a photoplethysmography (PPG) signal pertaining to the patient; analyzing the PPG signal to produce one or more PPG features; and applying at least one machine-learning (ML) model, trained to predict an inflammatory condition, on said one or more PPG features, to predict an inflammatory condition of the patient. Embodiments of the invention may subsequently produce at least one notification of the patient's inflammatory condition based on the prediction.

The inflammatory condition may include, for example a trajectory of inflammatory flare-up, a trajectory of inflammatory remission, and/or a failure of anti-inflammatory treatment.

Additionally, or alternatively, the notification may include, for example a suggested anti-inflammatory treatment and a change of treatment corresponding to the inflammatory status.

According to some embodiments, the at least one processor may receive, from at least one physiological sensor, at least one physiological signal pertaining to the patient. The at least one processor may analyze the at least one physiological signal, to produce one or more physiological features. The at least one processor may subsequently apply the at least one ML model on the one or more physiological features, to predict the inflammatory condition of the patient.

According to some embodiments, the at least one physiological sensor may be, or may include a thermometer, an accelerometer, a microphone, an ambient light sensor, a step counter, and a sleep quality sensor.

According to some embodiments, the at least one processor may receive, via a user interface (UI) at least one biomarker data element, representing a value of a biomarker pertaining to the patient. The at least one processor may apply the at least one ML model on the one or more biomarker data elements to predict the inflammatory condition of the patient.

According to some embodiments, the at least one biomarker data element may be, or may include a value of: Platelet count, Erythrocyte Sedimentation Rate, C-reactive protein concentration, Fecal calprotectin concentration, Blood viscosity, Perinuclear antineutrophil cytoplasmic antibodies, anti-Saccharomyces cerevisiae antibodies, Lactoferrin, Lipocalin-2, serum Albumin, serum Amyloid A, Ferritin, Fibronectin, Orosomucoid, α1-acid glycoprotein, Plasminogen, IL-1, IL-4, IL-5, and IL-10, TNF-α, IFN-α, IL-2, IL-6, IL-8, IL-12, IL-23, IL-23R, LIF-1, Rheumatoid factor, anti-cyclic citrullinated peptide, IL-12p40, Interferon alpha, IL-15, CCL3, CCL11, CXCL13, Calgranulin, VEGF, angiopotietin-2 and D-dimer value, blood transcriptomics, proteomics, metabolomics, microbiome and any combination thereof.

According to some embodiments, the at least one processor may receive, via the UI, at least one medical history data element. The at least one medical history data element may represent information pertaining to medical history of the patient. The at least one processor may apply the at least one ML model on the at least one medical history data element to predict the inflammatory condition of the patient, further based on the at least one medical history data element.

According to some embodiments, the at least one processor may apply the at least one ML model on the one or more PPG features by: applying at least one first ML model on said one or more PPG features, to predict at least one biomarker value; and applying at least one second ML model on said predicted biomarker value, to predict the inflammatory condition of the patient.

According to some embodiments, the at least one processor may train the at least one ML model to predict an inflammatory condition by: receiving a training dataset of one or more PPG features; receiving ground-truth labels of inflammation condition corresponding to the training dataset; and performing a back-propagation algorithm, to train the ML based model, based on the training set and labels.

Embodiments of the invention may include a system for assessment of an inflammatory status in a patient. Embodiments of the system may include a monitoring device, adapted to obtain a PPG signal pertaining to the patient; a non-transitory memory device, wherein modules of instruction code may be stored; and a processor associated with the memory device, and configured to execute the modules of instruction code. Upon execution of said modules of instruction code, the processor may be configured to: analyze the PPG signal to produce one or more PPG features; and applying at least one ML model, trained to predict an inflammatory condition, on said one or more PPG features, to predict an inflammatory condition of the patient.

According to some embodiments, the monitoring device may include one or more sensors, such as a PPG sensor and an accelerometer sensor. Additionally, the one or more sensors (e.g., the monitoring device) may include or may be associated with an electro-mechanical fixation system. The fixation system may be adapted to press or fasten the one or more sensors against a skin of the patient.

For example, the monitoring device (e.g., the one or more sensors) may be, or may be included in a non-invasive, wearable device or a patch device. Additionally, or alternatively, the monitoring device (e.g., the one or more sensors) may be implantable.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B and 1C are schematic diagrams, depicting application of a PPG sensor, and a corresponding PPG optical signal;

FIG. 2 is a block diagram, depicting a computing device which may be included in a system for non-invasive assessment of inflammatory conditions in patients according to some embodiments of the invention;

FIG. 3a is a block diagram, depicting a system for non-invasive assessment of inflammatory conditions in patients, according to some embodiments of the invention;

FIG. 3b illustrates a PPG signal obtained before and after application of pressure to reduce blood flow.

FIG. 3c illustrates an optical light sensor activated once a predetermined amount of shear forced has been applied.

FIG. 3d illustrates one embodiment of application of pressure to reduce blood flow by means of an inflatable cuff.

FIGS. 3e-3f illustrate a fixation element designed to press the one or more sensors/components against the user's skin, according to one embodiment of the present invention.

FIG. 4 is a graph, depicting an example of normalized PPG data or PPG signal, according to some embodiments of the invention;

FIG. 5 is a schematic diagram, depicting an example of an implementation of a system for performing non-invasive assessment of inflammatory conditions in patients, according to some embodiments of the invention;

FIG. 6 is a schematic diagram, depicting a classifier algorithm that may be implemented by a system for non-invasive assessment of inflammatory conditions in patients according to some embodiments of the invention;

FIG. 7 is a graph depicting Blood viscosity profile as a function of shear rate the present invention utilizes the fact that whole blood behaves as a non-Newtonian fluid and its viscosity is dependent on the amount shear rate exposed while sampling Thus, the illumination and PPG signal acquisition occur concomitantly with specified shear force applied that is measured by the accelerometer.

FIGS. 8-9 are a block diagram, depicting a system for non-invasive assessment of inflammatory conditions in patients, according to some embodiments of the invention;

FIG. 10 is a result of multi-parameter ML regression analysis Observed vs.

Predicted ESR values based on PPG signal non-invasive measurements of IBD patients. The model was trained on 75% from all records and the plots show results of predictions on the remaining 25%.

FIG. 11 is a result of multi-parameter ML regression analysis Observed vs.

Predicted CRP values based on PPG signal non-invasive measurements of IBD patients. The model was trained on 75% from all records and the plots show results of predictions on the remaining 25%.

FIG. 12 is a result of multi-parameter ML regression analysis Observed vs. Predicted PLT count values based on PPG signal non-invasive measurements of IBD patients. The model was trained on 75% from all records and the plots show results of predictions on the remaining 25%.

FIG. 13 is a principal component analysis (PCA) of a single patient. Dimensionality reduction using PCA performed to investigate the variability between PPG signals at different days and how they correlate to ESR levels. Most measurement days are well differentiated and clustered together. Notable differentiation between high and low ESR values.

FIG. 14 is a principal component analysis (PCA) of multiple patients. Dimensionality reduction using PCA performed to investigate the variability between PPG signals and how they correlate to ESR levels. A similar trend suggests that the variance between PPG signals is associated with both patients and ESR levels. Good separation between low, medium high ESR. Common PPG features are associated with ESR correlation.

### DETAILED DESCRIPTION OF THE PRESENT INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide non-invasive assessment and treatment of inflammatory conditions and status in patients.

It should be pointed out that although the above disclosure relates to the analysis of a PPG signal (as a stand alone or in combination with auxiliary sensors (e.g., accelerometer), it is within the scope of the present invention to provide a system and device that provide indication of at least one inflammatory status of a patient, comprising steps of
illuminating at least one location pertaining to the patient with at least one optical light beam characterized by at least one wavelength;
detecting at least one signal; said signal is at least a portion of at least one selected from a group consisting of the transmitted light beam, the absorbed light beam, the reflected light beam and any combination thereof; and,
analyzing said at least a portion of said signal as a function of time thereby indicating said inflammatory status of said patient;
   wherein said step of analyzing at least a portion of said signal additionally comprising step of: (a) extracting at least one feature; (b) analyzing at least one trend of said at least one feature as a function of time; and, (c) correlating said at least one trend with at least one biomarker indicative of said at least one inflammatory status.

It further within the scope of the present invention where detecting is performed by at least one sensor selected from a group consisting of photodiode and/or laser light source.

It is further within the scope of the present invention, where the wavelength/s used is/are as follows:

For photodiode:
Visible light 400-700 nm
And IR from 1 mm to 700 nm

For the laser light source:
the whole diode laser spectrum can be used in medical applications of diode lasers cover, starting from 200 nm ultraviolet and violet DLs used for sterilization and some surgery applications, through photodynamic therapy (PDT) in the visible wavelength range at 630-690 nm, to longer wavelengths.

Thus, it is emphasized that where there is reference to a PPG signal—it could also relate to any other signal produced by a light sources.

The term “about” as used herein includes, a range of 25% below or above the referred value.

The term “chronic inflammatory diseases” as used herein includes, but is not limited to, Inflammatory Bowel Diseases (IBD) including ulcerative colitis (UC) and Crohn's disease (CD), Rheumatological diseases, Rheumatoid Arthritis (RA), Psoriatic Arthritis (PsA), Spondyloarthritis (SpA) Psoriasis, Chronic Obstructive Pulmonary Disease (COPD), Asthma, Systemic Lupus Erythematosus (SLE), Multiple Sclerosis (MS), Vasculitis, malignant diseases, cardiovascular diseases, Immune-Mediated Heart Diseases, Infection-Related Immune-Mediated Diseases and other inflammatory chronic diseases. These diseases are characterized by periods of remissions and flare ups. The term “flare-ups” as used herein refers to when a disease is active and showing full symptoms, which lead to deterioration, impact on quality of life and work productivity of the patient. It is a devastating situation for the patient. furthermore, early detection and treatment may be of therapeutic importance because treatment of disease before its full blown exacerbation and according to disease biomarkers may be more effective, shorter and prevent inflammation-related tissue damage.

The term “flare” as used herein may refer to a condition in which a disease is active and showing full symptoms, which may lead to deterioration, and may have an impact on quality of life and work productivity of the patient. Early detection and treatment of flares may be of therapeutic importance: treatment of disease before a full-blown exacerbation, according to disease biomarkers may be more effective, shorter and prevent inflammation-related tissue damage.

The term “biomarker” as used herein, may refer to any measurable indicator of a biological state or condition. Biomarkers may be measured and/or evaluated to examine biological and physiological processes, pathologic processes, inflammation, and/or pharmacologic responses to a therapeutic intervention. Non-limiting examples of biomarkers may include: Platelet count (PLT), Erythrocyte Sedimentation Rate (ESR), C-reactive protein (CRP) concentration, Fecal calprotectin concentration, Perinuclear antineutrophil cytoplasmic antibodies (PANCAs), anti-Saccharomyces cerevisiae antibodies (ASCAs), Lactoferrin, Lipocalin-2, serum Albumin, serum Amyloid A, Ferritin, Fibronectin, Orosomucoid (α1-acid glycoprotein), Plasminogen, IL-1, IL-4, IL-5, and IL-10, TNF-α, IFN-α, IL-2, IL-6, IL-8, IL-12, IL-23, IL-23R, LIF-1, Rheumatoid factor, anti-cyclic citrullinated peptide, IL-12p40, Interferon alpha, IL-15, CCL3, CCL11, CXCL13, Calgranulin, VEGF, angiopotietin-2 and D-dimer value, blood transcriptomics, proteomics, metabolomics, microbiome and any combination thereof.

The term “Inflammatory Bowel Disease (IBD)” as used herein, may refer to a group of inflammatory conditions of the colon and small intestine. IBD describes disorders involving long-standing (chronic) inflammation of tissues in your digestive tract. Types of IBD include:
Ulcerative colitis. This condition involves inflammation and sores (ulcers) along the lining of your large intestine (colon) and rectum.
Crohn's disease. This type of IBD is characterized by inflammation of the lining of your digestive tract, which often can involve the deeper layers of the digestive tract. Crohn's disease most commonly affects the small intestine. However, it can also affect the large intestine and uncommonly, the upper gastrointestinal tract.

The term/feature “RR”, “PI”, “DIT”, “RAI”, “RDI”, “NRDI”, “BOA”, “MSL”, “LF”, “HF”, “LF/HF”, “DC”, “AC”, “peripheral index” are as defined in Table 1.

The term “photoplethysmogram (PPG)” as used herein, may refer to an optically obtained plethysmogram that can be used to detect blood volume changes in the microvascular bed of tissue. A PPG is often obtained by using a pulse oximeter which illuminates the skin and measures changes in light absorption. A conventional pulse oximeter monitors the perfusion of blood to the dermis and subcutaneous tissue of the skin.

With each cardiac cycle the heart pumps blood to the periphery. The change in volume caused by the pressure pulse is detected by illuminating the skin with the light from a light-emitting diode (LED) and then measuring the amount of light either transmitted or reflected to a photodiode. Each cardiac cycle appears as a peak.

Because blood flow to the skin can be modulated by multiple other physiological systems, the PPG can also be used to monitor breathing, hypovolemia, and other circulatory conditions. Additionally, the shape of the PPG waveform differs from subject to subject, and varies with the location and manner in which the pulse oximeter is attached.

Thus, the present invention utilizes PPG signal and analysis thereof to provide indication as to the assessment and treatment of inflammatory conditions and status in patients. Embodiments of the method may include receiving a photoplethysmography (PPG) signal pertaining to the patient; analyzing the PPG signal to produce one or more PPG features; and applying at least one machine-learning (ML) model, trained to predict an inflammatory condition, on said one or more PPG features, to predict an inflammatory condition of the patient. Embodiments of the invention may subsequently produce at least one notification of the patient's inflammatory condition based on the prediction.

Embodiments of the invention include a system for assessment of an inflammatory status in a patient. Embodiments of the system may include a monitoring device, adapted to obtain a PPG signal pertaining to the patient; and a processor configured to analyze the PPG signal to produce one or more PPG features; and applying at least one ML model, trained to predict an inflammatory condition, on said one or more PPG features, to predict an inflammatory condition of the patient.

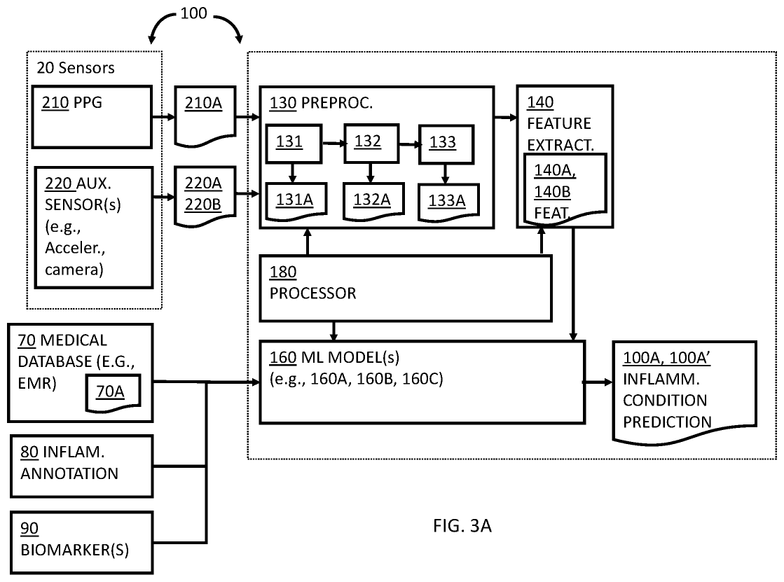

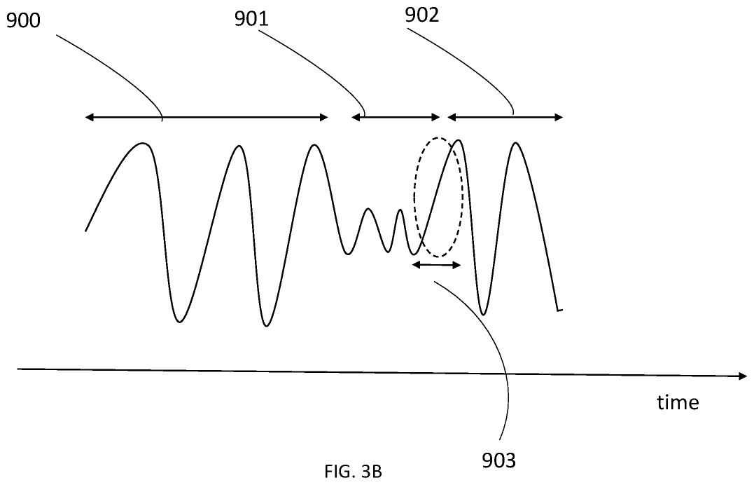

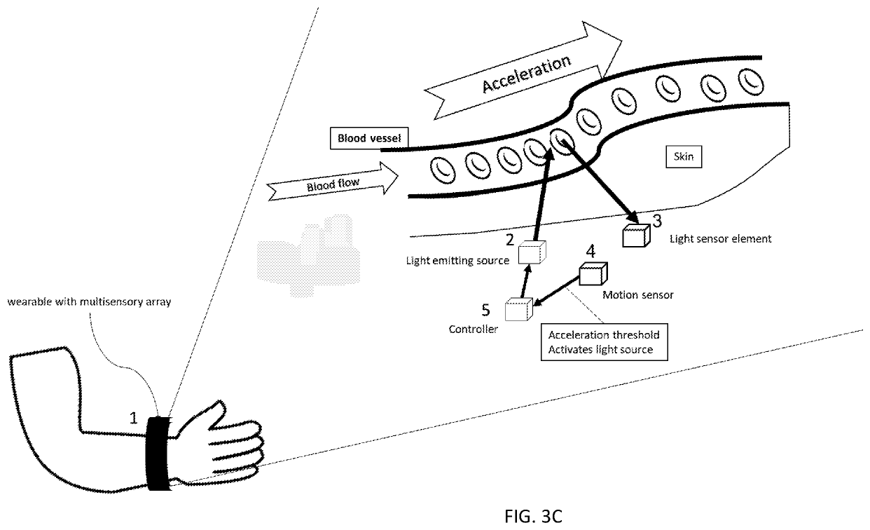

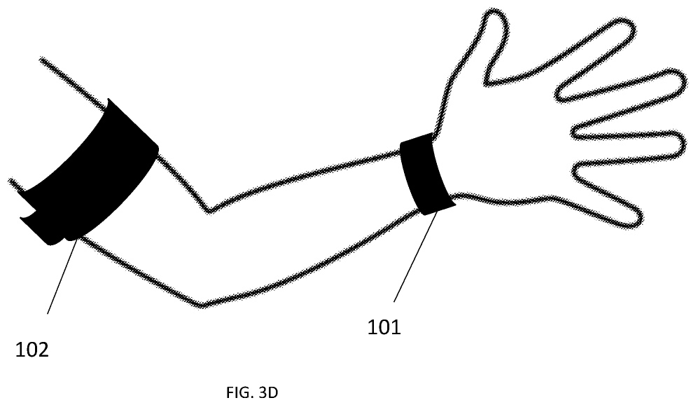

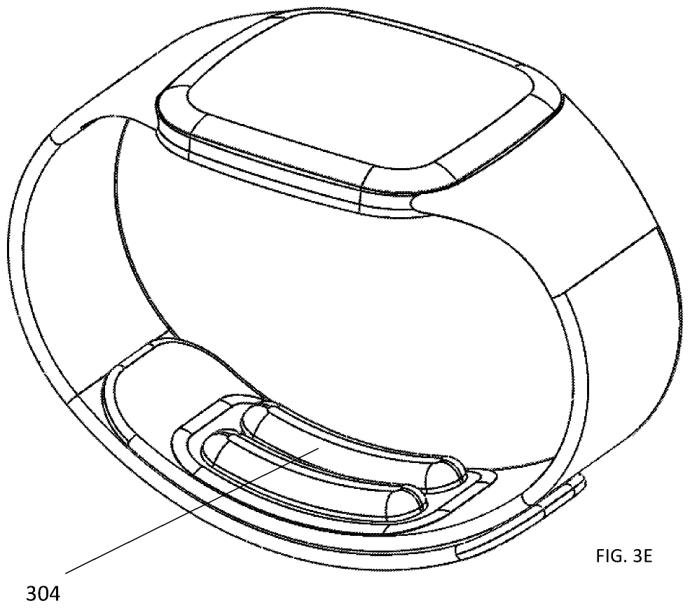

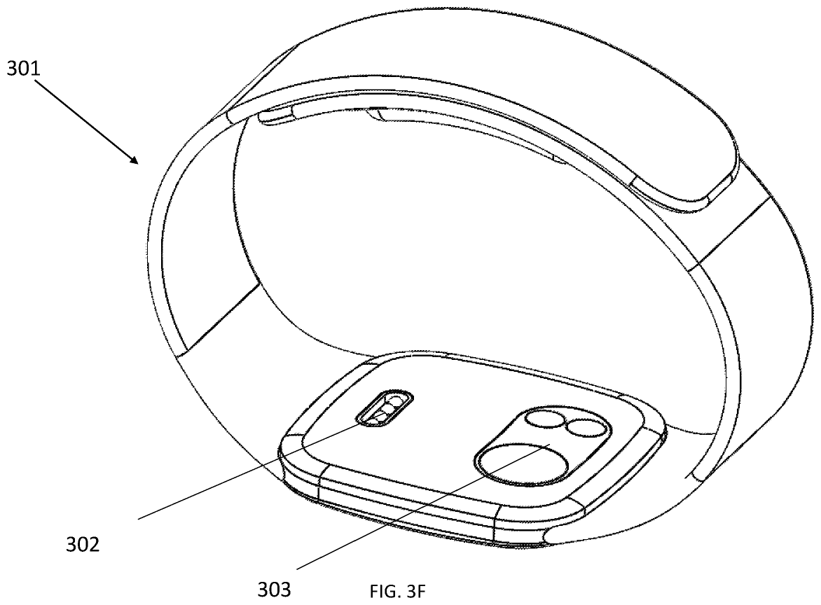

According to some embodiments, the monitoring device may include one or more sensors, such as a PPG sensor and an accelerometer sensor. Additionally, the one or more sensors may include or may be associated with an electro-mechanical fixation system (see FIGS. 3e-3f). The fixation system may be adapted to press or fasten the one or more sensors against a skin of the patient.

According to some embodiments, the sensor(s) includes at least one selected from a group consisting of an accelerometer, a camera, a microphone, a step counter, a sleep quality sensor and the like, which may be configured to obtain data pertaining to movement of the patient and or the patient's organ upon which the PPG sensor is associated with (e.g., a finger, a wrist).

Alternatively of additionally, the sensor(s) includes at least one selected from a group consisting one or more ambient sensors, such as an ambient light sensor, a thermometer, and the like, which may be configured to obtain data pertaining to the ambience (e.g., temperature, ambient light) of the patient, and/or a temperature of the patient.

According to some embodiments the sensors may be, or may be included in a non-invasive, wearable device or a patch device. Additionally, or alternatively, the monitoring device (e.g., the one or more sensors) may be implantable.

According to one embodiment of the present invention the present invention can be utilized to provide indication as to the status of inflammatory conditions selected from a group consisting of immune mediated diseases; said immune mediated diseases being selected from a group consisting of Inflammatory Bowel Diseases (IBD) including ulcerative colitis (UC) and Crohn's disease (CD), Rheumatological diseases, Rheumatoid Arthritis (RA), Psoriatic Arthritis (PsA), Spondyloarthritis (SpA), Psoriasis, Chronic Obstructive Pulmonary Disease (COPD), Asthma, Systemic Lupus Erythematosus (SLE), Multiple Sclerosis (MS), Vasculitis, malignant diseases, cardiovascular diseases, Immune-Mediated Heart Diseases, Infection-Related Immune-Mediated Diseases and any combination thereof.

As noted above, it is within the core concept of the present invention to provide a system and method that combines the signal of a PPG sensor with at least one additional sensor (e.g., accelerometer/ECG) to provide indication/assessment of inflammatory condition and status and determination of the disease activity state in patients.

The analysis of the combined utilized sensors is based on at least one of the following:

a. Blood Flow Rheology/Haemorheology:

blood flow rheological properties may be used to assess erythrocytes aggregability and deformability, vascular resistance, plasma viscosity and hematocrit as reliable measures of acute phase inflammation. The blood flow rheological properties may be based on the tracking of changes from a predefined/pre-measured baseline. Such changes may be indicative of a subclinical inflammation before a flare-up occurs.

Notably, it is a measurable tool for therapy efficiency in chronic inflammatory diseases.

The present invention utilizes the fact that whole blood behaves as a non-Newtonian fluid and its viscosity is dependent on the amount shear rate exposed while sampling, as known in the art. Thus, the illumination and PPG signal acquisition may occur concomitantly with specified shear force applied that is measured by the accelerometer. In other words, the signal acquisition is performed in a particular timing, synchronized with the needed acceleration (induced shear force on the predetermined body part on which the optical sensor, e.g., PPG, is placed) that is measured by the accelerometer.

The blood flow rheology/haemorheology algorithm may be used by system 100 to assess blood rheology properties by analyzing changes in the PPG signal obtained from illuminating blood vessels and skin of the subject at rest and after applying shear force on the blood in the blood vessels.

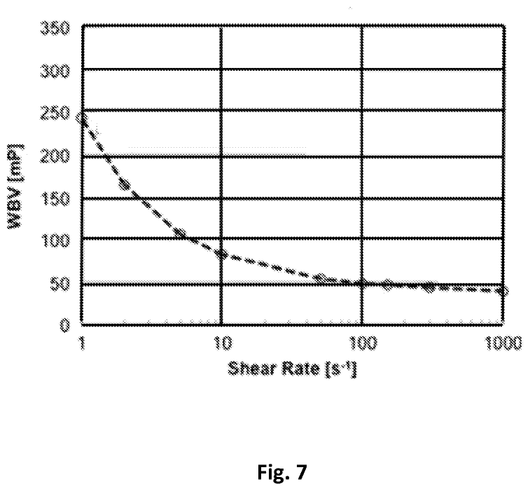

Reference is now made to FIG. 7, which is a graph, published in the article “Endothelial Shear Stress and Blood Viscosity in Peripheral Arterial Disease”; Young I. Cho & Daniel J. Cho & Robert S. Rosenson; Curr Atheroscler Rep (2014) 16:404; DOI 10.1007/s11883-014-0404-6) depicting blood viscosity profile as a function of shear rate. As shown in FIG. 7, the present invention utilizes the fact that whole blood behaves as a non-Newtonian fluid and its viscosity is dependent on the amount shear rate exposed while sampling, as known in the art. Thus, the illumination and PPG signal acquisition may occur concomitantly with specified shear force applied that is measured by the accelerometer.

It should be noted that said applying shear force is performed by maneuvering said at least one location pertaining to the patient at at least one selected from a group consisting of predetermined speed, predetermined rhythm, for a predetermined period of time and any combination thereof.

According to some embodiments, controller 180 of FIG. 3a may control the optical sensor 210 and/or auxiliary sensor 220 (e.g., an accelerometer) to measure the mechanical effect and response of the blood flow as obtained from the collected PPG signal during acceleration of blood in the vessels. This approach may allow detection of changes in blood viscosity, blood flow and erythrocyte aggregation, all of which are markers for an active inflammation phase. Embodiments of the invention may thus include an improvement over currently available methods and systems of monitoring of haemorheology properties.

Thus, it is one object of the present invention to provide at least one blood flow rheological parameter, wherein said at least one blood flow rheological parameter is adapted to indicate at least one selected from a group consisting of erythrocytes aggregation, plasma viscosity and any combination thereof. It is further within the scope of the present invention where the blood flow rheological parameter indicates said inflammatory status.

As indicated above, it is known in the literature that the blood's viscosity is indicative to inflammation status, thus providing a baseline pattern of at least one blood flow rheological parameter (specifically, the viscosity thereof) for each of said inflammatory status, will facilitate indication of a change of said inflammatory status once a deviation above a predetermined threshold from said baseline pattern. More specifically, monitoring a baseline pattern of the PPG signal of at least one blood flow rheological parameter (specifically, the viscosity thereof) for each of said inflammatory status, will facilitate indication of a change of said inflammatory status once a deviation above a predetermined threshold from said baseline pattern.

According to another embodiment, the optical sensor 210 (namely, the PPG sensor) and/or auxiliary sensor 220 (namely, the accelerometer) are configured to measure the mechanical effect and response of the blood flow as obtained from the collected PPG signal during acceleration of blood in the vessels. This approach may allow detection of changes in blood viscosity, blood flow and erythrocyte aggregation, all of which are markers for an active inflammation phase. Embodiments of the invention may thus include an improvement over currently available methods and systems of monitoring of haemorheology properties.

Thus, it is one object of the present invention to provide at least one blood flow rheological parameter, wherein said at least one blood flow rheological parameter is adapted to indicate at least one selected from a group consisting of erythrocytes aggregation, plasma viscosity and any combination thereof. It is further within the scope of the present invention where the blood flow rheological parameter indicates said inflammatory status.

As indicated above, it is known in the literature that the blood's viscosity is indicative to inflammation status, thus providing a baseline pattern of at least one blood flow rheological parameter (specifically, the viscosity thereof) for each of said inflammatory status, will facilitate indication of a change of said inflammatory status once a deviation above a predetermined threshold from said baseline pattern. More specifically, monitoring a baseline pattern of the PPG signal of at least one blood flow rheological parameter (specifically, the viscosity thereof) for each of said inflammatory status, will facilitate indication of a change of said inflammatory status once a deviation above a predetermined threshold from said baseline pattern.

According to another embodiment of the present invention, the base unit (e.g., the PPG sensor) and the accelerometer can transmit the data to a mobile device or to a cloud-based data storage mean.

In the cloud, the analysis of the signal is performed and the results thereof is sent to the medical team.

It is within the scope of the present invention to provide means adapted for:
i. momentarily, for a predetermined period of time, reducing blood flow from reaching said at least one location pertaining to said patient; thereby said at least one PPG signal (or any other optical light signal) is attenuated;
ii. after said predetermined period of time, enabling said blood flow to reach said at least one location pertaining to said patient; thereby said at least one PPG signal (or any other optical light signal) is regained.

By analyzing at least one feature of the attenuated signal versus the regained signal, said inflammatory status is provided based on said analysis.

According to one embodiment of the present invention, the processor is measuring the amount of time needed for said at least one PPG signal (or any other optical light signal) to regain; thereby providing the inflammatory status, based on said amount of time needed for said at least one PPG signal (or any other optical light signal) to regain.

According to another embodiment of the present invention, the feature is selected from a group consisting of the amount of time needed for said at least one PPG signal to regain, the rate at which said at least one PPG signal (or any other optical light signal) is regained, the intensity of said attenuated PPG signal (or any other optical light signal), the integral of the signal as a function of time, the derivative of the signal as a function of time and any combination thereof.

According to one embodiment of the present invention the momentarily reducing/preventing blood flow is performed by applying pressure on at least one predetermined location. According to another embodiment of the present invention, the momentarily reducing/preventing blood flow is performed by means of at least one cuff encircling the at least one predetermined location (from which the PPG signal is measured).

According to one embodiment of the present invention the momentarily reducing/preventing blood flow is performed at least one predetermined location is selected from a group consisting of armpit, forearm, finger, leg, ankle, wrist, ear and any combination thereof.

According to another object of the present invention, at least one vibration (by means of at least one vibrating element) is applied to detect the inflammatory status.

According to this embodiment, the blood viscosity during inflammation is characterized by a substantially different characteristics when compared with the blood viscosity when without inflammation. Thus, application of vibration and detecting the signal resulted from said vibration application will provide information as to the blood viscosity and therefrom—the inflammatory state.

Analysis of the signal for feature such as the amount of time needed for said at least one PPG signal to regain; the advancement rate of said vibrations from said at least one location along an advancement path, the intensity thereof and any combination thereof will provide information as to the inflammatory status of the patient.

Thus, according to this embodiment, the method, additionally comprising steps of
a. applying, for a predetermined period of time, at least one vibration on said at least one location; thereby said at least one PPG signal is changed;
b. after said predetermined period of time, removing said applied vibration; thereby said at least one PPG signal is regained;
c. analyzing at least one feature of at least one selected from a group consisting of the changed signal, the changed signal versus the regained signal and any combination thereof;
   wherein said inflammatory status is provided based on said analysis.

It is another object of the present invention to provide the method as defined above, wherein said feature is selected from a group consisting of the amount of time needed for said at least one PPG signal to regain; the advancement rate of said vibrations from said at least one location along an advancement path, the intensity thereof and any combination thereof.

Alternatively, the system will include a vibration element that will, for a predetermined period of time, apply vibration to a predetermined body location and at least one feature of the signal will be analyzed. The feature could be the amount of time needed for said at least one PPG signal to regain; the advancement rate of said vibrations from said at least one location along an advancement path, the intensity thereof and any combination thereof.

Reference is now made to FIG. 3b illustrating a PPG signal measurement 900 before application of pressure to momentarily reduce/prevent blood flow (by, e.g., a cuff applying pressure) and thereafter.

It should be noted that although PPG signal is disclosed, it is within the scope of the present invention where any other optical light signal is included.

As seen in FIG. 3b, once the blood flow is reduced, the PPG signal is attenuated (see numerical reference 901).

After the pressure is removed and the blood is allowed to flow, the PPG signal is regained, see numerical reference 902.

Analysis of at least one feature of the attenuated signal versus the regained signal enables the provision of the inflammatory status. Such feature could be the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

According to one embodiment, the time it takes for the PPG signal to regain its normal values (see numerical reference 903 and encircled by a dotted circle) is an indication of the viscosity of the blood. Thus, one can indicate the inflammatory condition.

As disclosed above, other features could be used as indicator for blood flow rheological properties (e.g., the viscosity of the blood) and thus indicative of the inflammatory status.

It should be noted that it is within the scope of the present invention where the cuff is an inflatable one, such that when the same is inflated, pressure is applied on said at least one predetermined location (to reduce/prevent blood flow) and once the cuff is deflated, said pressure is removed to allow flood flow.

Alternatively, inducing acceleration of blood by applying shear force on the same could also, in a similar manner, provide indication as to the viscosity of blood flow and indication of the inflammatory condition. In other words, analyzing changes in said at least one PPG signal before and after said step of applying shear force on blood will provide indication as to the inflammatory status of the patient.

Reference is now being made to FIG. 3c illustrating such an embodiment. As seen in the figure, a wearable device, 1, have at least one sensor (optical light emitting sensor), preferably a plurality of sensors. When acceleration is induced on the blood flow (by e.g., movement induced by the patient), the accelerometer, 4, detects such motion and when the same accelerates above a predetermined threshold the controller 5 enables the optical light emitting sensor 2 to be activated and emit at least one optical light beam (at at least one predetermined wavelength); thereby light detection (either of the transmitted light, absorbed or reflected light) is enabled by at least one light sensor 3.

According to another embodiment of the present invention, a reference signal as a function of time is obtained.

According to one embodiment, the reference signal is at least one selected from a group consisting of the transmitted light beam, the absorbed light beam, the reflected light beam and any combination thereof as a function of time, of at least one selected from a group consisting of said patient with a known inflammatory status, of at least one group of patients of with known inflammatory status, an average of at least one group of patients of with known inflammatory status, and any combination thereof. Thus, the reference signal serves as a base line for comparison with the detected signal.

According to one embodiment, the known inflammatory status is selected from a group consisting of a flare-up, a remission, a healthy pattern and any combination thereof.

According to one embodiment, the analysis of the signal as a function of time additionally comprising step of comparing and identifying deviations of the same with said at least one reference signal as a function of time.

It should be noted that it is within the scope of the present invention where the reference signal is taken whenever is needed or required. For example, the reference signal could be taken when the patient is at rest and not when he is under physical activity.

Reference is now to FIG. 3d illustrating an embodiment of application of pressure to reduce blood flow by means of an inflatable cuff. As seen in the figure, an inflatable cuff 102 is placed on the arm while the device of the present invention 102 (including the optical sensor, e.g., PPG sensor) is placed further downstream. Once a measurement is desired, the inflatable cuff 102 is inflated, thus, applying pressure on the arm (and the blood flow); thereby the blood flow is reduced to alter the optical signal sensed by the optical sensor in wearable device 102 (e.g., the PPG signal). Once the pressure is relieved (namely, by deflating the inflatable cuff), the optical signal (namely, the PPG signal) would regain its original form.

As described above, analysis of features of the altered signal relative to the original (or regain one), one could provide indication as to the inflammatory status of the patient.

(B) Pulse Rate and (C) Pulse Wave Velocity Obtained from the PPG Signal:

It has been shown that in some chronic inflammatory diseases (such as IBD) arterial stiffness is associated with active inflammation. Increased arterial stiffness is dependent upon inflammation and reduced by immunomodulatory drugs. Specifically, during active disease/inflammation arterial stiffness is increased and may lead to measurable changes in pulse wave velocity (PWV). PWV is the speed of a pressure pulse propagating along the arterial wall and can be calculated from pulse transit time (PTT). PTT is the time between two pulse waves propagating on the same cardiac cycle from two arterial sites. PWV is the speed at which the forward pressure wave is transmitted from the aorta through the vascular tree, and is calculated by measuring the time taken for the arterial waveform to pass between two points a measured distance apart, and involves taking readings from the two sites simultaneously, or gating separate recordings to a fixed point in the cardiac cycle (e.g., the R-wave of the ECG, the PPG signal, pressure or flow signals, or a combination of both). As known in the art, the relationship between PWV and arterial stiffness may be the Moens Korteweg equation: PWV=√(E×h/2rp), where: E-Young's modulus of elasticity of wall material; h=wall thickness of vessel; r-inside radius of vessel; and ρ=density of blood.

According to some embodiments, system 100 and the algorithms used thereby may use continuous measurements of PWV, and may track changes in arterial stiffness to assess and predict, together with other parameters, the disease state trajectory. Accordingly, arterial stiffness is non-invasively assessed according to pulse wave velocity measurements obtained from the PPG signal. Moreover, some studies indicate that changes in radial pulse wave velocity may occur with high blood viscosity.

According to some embodiments, system 100 may determine PWV by PPG signal or with a combination of PPG and Electrocardiogram (ECG) signals.

The term “Electrocardiogram” refers hereinafter to a record of the heart's electrical activity.

By employing a pair of sensors 20 (either 2 PPG sensors or one PPG and one ECG sensor), the amount of time taken by the pulse wave to traverse an arterial segment can be determined and used to calculate the PWV, according to the following equation: PWV=L/PTT, where L is the distance between the two measurement points; and PTT is the transit time of the pulse wave (e.g., the time delay between the two signals).

### (D) Biomarker Analysis & Therapeutics Levels:

Therapeutics and biomarkers levels help to define the patient's clinical status and disease activity status. Therapeutics levels are important to determine therapy efficiency and its impact on disease activity. Biomarkers levels (such as inflammatory biomarkers) are important to determine the disease activity status. Such data may be obtained from measurements of saliva, blood and/or urine samples using spectroscopy analysis and/or by non-invasive optical measurements of samples thereof.

According to this embodiment of the present invention analysis of blood or saliva samples will be used to track biomarkers or therapeutics levels in addition to the non-invasive PPG measurements at home.

### (F) Inflammatory Clinical Behaviorome:

The term behaviorome refers hereinafter to capturing, analysis and interpretation of human behavior as a determinant of health. Thus, behaviorome may be used herein to refer to a set of digital markers (e.g., step counts, motion sensors, geolocators, sleep patterns; indoor and outdoor duration patterns; eating habits/appetite patterns; number of daily visits in the restrooms, motion/ambient light/humidity sensors, geolocators, UV sensors, heart rate sensor, daily steps counter patterns, body temperature sensors, humidity sensor, ambient light sensor, data inserted by the patient himself (e.g., symptoms the patient has, nutrition etc.)) that can be collected, and may reflect a patient's status, such as fatigue that is correlated with increased inflammation.

This is a collection of activities and behaviors that can be collected and analyzed using the sensors array 20 and passive data collection. This sub-algorithm may analyze all the collected data and may utilize machine learning tools and heuristic rules to identify patterns and hidden patterns that correlate with disease activity.

In certain embodiments, the data may be collected from various sensors 20 that are able to capture daily activities such as: motion sensors, geolocators, UV sensors, heart rate sensor, body temperature sensors, humidity sensor, ambient light sensor etc. Accordingly, in some embodiments, the activity types and behaviors may be collected and analyzed to generate the “inflammatory clinical behaviorome”.

According to some embodiments of the invention, system 100 may utilize any combination of the abovementioned sub-algorithms/variables. The combination of sub-algorithms/variables as well as their importance and relevance (i.e., impact) varies between different diseases; between patients with the same condition/disease; and between different stages of disease/condition within a specific patient (e.g., between flare-up and remission).

According to some embodiments, the activity types and behaviors include, but not limited to overall all activity patterns; daily steps counter patterns; body temperature patterns; sleep patterns; indoor and outdoor duration patterns; eating habits/appetite patterns; number of daily visits in the restrooms, etc., and any combination thereof.

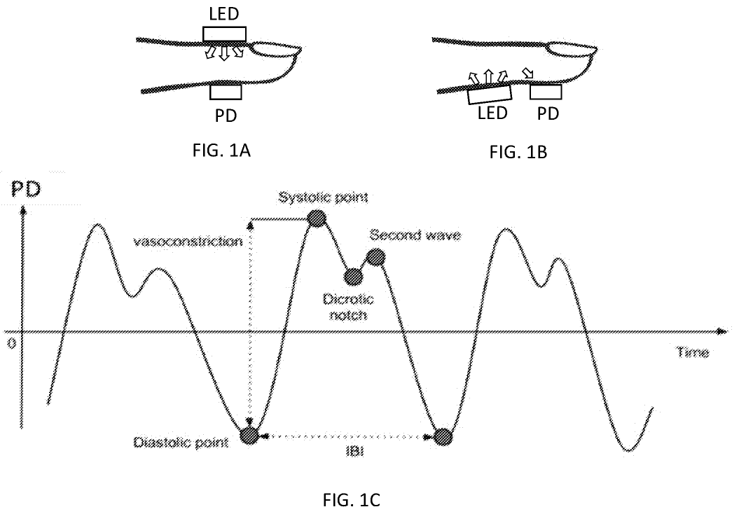

Reference is now made to FIGS. 1A, 1B and 1C depicting application of a PPG sensor, and a corresponding PPG optical signal, as known in the art. The PPG sensor may be attached to a patient's organ (e.g., a finger, as in the depicted example), and may be used to detect blood volume changes in the microvascular bed of tissue. In some embodiments, the PPG sensor may detect the change in blood vessel volume, caused by the heart, by illuminating the skin by a light-emitting diode (LED) in the red or infra-red spectrum, and then measuring the amount of light either transmitted (e.g., as in the example of FIG. 1A) or reflected (e.g., as in the example of FIG. 1B) to a photodiode. Based on these measurements, the PPG sensor may produce an optical signal (e.g., as depicted in the example of FIG. 1C) representing the transmission or reflection of light. Embodiments of the invention may be adapted to utilize the PPG signal to evaluate or assess an inflammatory condition of a patient, as elaborated herein.

In certain embodiments, the main monitoring unit may further include an electro-mechanical fixation system designed to press the one or more sensors/components against the user's skin. For example, the fixation can be done by an inflating balloon or by a spring mechanism or by a shape-memory alloy that are activated by an actuator. In certain embodiments, the pressing of the sensors/components against the user's skin is carried out in a cyclic manner according to measurements, e.g., immediately before measurement, the electro-mechanical fixation system presses the sensors against the patient's skin to obtain contact, and as soon as the measurement is complete, the pressure is released.

Reference is now made to FIGS. 3e-3f illustrating such a device with a fixation system for pressing the one or more sensors/components against the user's skin. In this case the device is a wearable device.

As seen in the figures, the wearable device 301 comprising at least one optical sensor 302 and at least one optical source 303. Also illustrated are the fixation element, 304, where in this case are inflatable elements, adapted to, when inflated, to ensure the wearable device is pressed on the patient's skin.

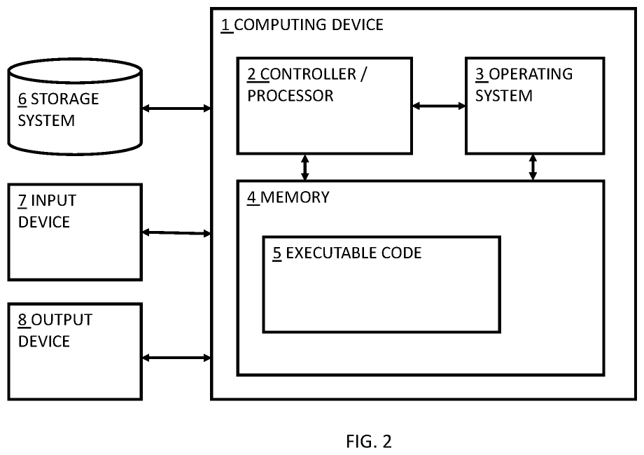

Reference is now made to FIG. 2, which is a block diagram depicting a computing device, which may be included within an embodiment of a system for non-invasive assessment of inflammatory conditions in patients, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may assess inflammatory conditions in patients as further described herein. Although, for the sake of clarity, a single item of executable code 5 is shown in FIG. 2, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data representing measurements, performed by one or more sensors, and pertaining to one or more patients may be stored in storage system 6 and may be loaded from storage system 6 into memory 4 where it may be processed by processor or controller 2. In some embodiments, some of the components shown in FIG. 2 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

A neural network (NN), or artificial neural network (ANN), such as an ANN implementing a machine learning (ML) model such as a support-vector machine (SVM) model, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

Reference is now made to FIG. 3a, which is a block diagram depicting a system 100 for performing non-invasive assessment of inflammatory conditions in patients, according to some embodiments of the invention. System 100 may be implemented as a software module, a hardware module, or any combination thereof.

For example, system 100 may be or may include a computing device such as element 1 of FIG. 2 and may include at least one processor 180 (such as processor 2 of FIG. 2). Processor 180 may be adapted to execute one or more modules of executable code (e.g., element 5 of FIG. 2) to perform non-invasive assessment of inflammatory conditions in patients, as further described herein.

Arrows in FIG. 3a may represent flow of data among to or from system 100, and/or between modules of system 100. It may be appreciated that some arrows have been omitted here for the purpose of clarity.

According to some embodiments, system 100 may be adapted to identify and/or alert when an upcoming flare-up is about to occur, before the actual manifestation of symptoms, thereby enabling preliminary treatment and/or treatment adjustments, as well as patient self-management. Such alerts may lead to “deep remission”, as commonly referred to in the art, as a clinical goal, and may lead to reduction of active disease symptoms.

Additionally, system 100 may be adapted to track treatment effect during flare-ups and remission. Treatment may include administration of pharmacological agents such as anti-inflammatory drugs, steroids, immunosuppressives and treatment with anti-inflammatory monoclonal antibodies. Early reliable detection of changes in the inflammatory status may indicate a correct treatment choice, or alternatively alert for a need to change the therapeutic regimen, and avoid applying futile treatments, which may also be associated with significant side effects.

According to some embodiments, and as depicted in FIG. 3a, system 100 may include, or may be electronically or communicatively connected to a monitoring device that may include one or more sensors 20. The one or more sensors 20 of the monitoring device may be, or may include for example a PPG sensor 210, which may be attached to a patient's organ (e.g., a finger) and may produce an electric PPG signal 210A. For example, electric PPG signal 210A may represent an optical signal obtained by PPG sensor 210, as elaborated herein in relation to FIGS. 1A-1C. System 100 may store at least one representation of PPG signal 210A on at least one memory device (e.g., memory 4 of FIG. 2) or storage device (e.g., storage 6 of FIG. 2) for further analysis, as elaborated herein.

Additionally, the one or more sensors 20 may be, or may include one or more auxiliary physiological sensors 220, including for example an accelerometer, a camera, a microphone, a step counter, a sleep quality sensor and the like, which may be configured to obtain data 220A pertaining to movement (e.g., acceleration) of the patient and or the patient's organ (e.g., a finger, a wrist). Additionally, or alternatively, the one or more sensors 20 may be, or may include one or more ambient sensors, such as an ambient light sensor, a thermometer, and the like, which may be configured to obtain data 220B pertaining to the ambience (e.g., temperature, ambient light) of the patient, and/or a temperature of the patient.

According to some embodiments, the monitoring device (e.g., the one or more sensors 20) may include an electro-mechanical fixation system, adapted to fasten or press said one or more sensors against a skin of the patient. For example, the monitoring device (e.g., the one or more sensors 20) may be a non-invasive wearable device, or a patch device.

Additionally, or alternatively, the monitoring device (e.g., the one or more sensors 20) may be implantable.

According to some embodiments, system 100 may include a preprocessing module 130, configured to receive or obtain a PPG signal 210A pertaining to a patient from PPG sensor 210. Additionally, preprocessing module 30 may be configured to receive or obtain a data element or signal 220A from one or more auxiliary sensors 220, representing movement of the patient and/or movement of an organ of the patient to which PPG sensor 210 is attached. Preprocessing module 130 may perform one or more actions of signal and/or data processing on PPG signal 210A as elaborated herein, to produce a digitized, normalized version of PPG signal 210A, which is herein referred to as “normalized PPG data” 130A.

According to some embodiments, preprocessing module 130 may include at least one signal processing module 131, as known in the art. Signal processing module 131 may include, for example a noise filter, adapted to improve a signal-to-noise ratio (SNR) of the incoming PPG signal 210A. Additionally, signal processing module 131 may include an adaptive gain module, configured to apply adaptive gain on the incoming PPG signal 210A. Additionally, signal processing module 310 may include an analog to digital (A2D) module, adapted to produce a digital, sampled representation of PPG signal 210A. Signal processing module 131 apply these signal processing to produce a first processed version 131A of PPG signal 210A.

Additionally, or alternatively, preprocessing module 130 may include an artifact removal module 132, adapted to remove artifacts, or sequences may include artifacts in PPG signal 210A. The term “artifact” may be used in this context to indicate at least a portion of PPG signal 210A that may compromise the PPG measurement. For example, movement of the patient may affect PPG signal 210A, and may compromise the integrity of PPG signal 210A as representing a condition of the patient.

According to some embodiments, artifact removal module 132 may receive auxiliary sensor data 220A, from at least one auxiliary or physiological sensor 220, and may determine therefrom an extent of movement of the patient.

For example, auxiliary sensor 220 may be an accelerometer 220 attached to the patient. Artifact removal module 132 may obtain from accelerometer 220 data 220A pertaining to movement of the patient and or movement of PPG sensor 210. Artifact removal module 132 may detect whether the patient has moved PPG sensor 210, and/or the extent and direction of a patient's movement of PPG sensor 210, based on auxiliary sensor data 220A. Artifact removal module 132 may subsequently remove or omit a section of processed PPG data 131A that corresponds to this movement, to create a second processed version 132A of PPG data 210A. In other words, second processed version 132A may be devoid from movement artifacts, so as not to take the movement artifacts into consideration during assessment of the patient's condition.

In another example, auxiliary sensor 220 may be a camera pointed at or monitoring the patient. Auxiliary sensor data 220A may be a video stream depicting at least part of the patient's body. In such embodiments, artifact removal module 320 may obtain from camera 220 data 220A (e.g., a video stream) depicting or pertaining to movement of the patient and/or movement of PPG sensor 210. Artifact removal module 320 may apply any appropriate image processing algorithm as known in the art to determine extent of movement of PPG sensor 210 by the patient. Subsequently, artifact removal module 320 may label or mark one or more portions of PPG signal 210A (or first processed version 131A) as including artifacts of movement, to exclude such portions from further processing or calculation, as elaborated herein.

According to some embodiments, preprocessing module 130 may include a normalization module 133, adapted to receive at least one version of PPG signal (e.g., 210A, 131A, 132A), and normalize the at least one version of PPG signal, to enable extraction of features therefrom, as elaborated herein. It is within the scope of the present invention where the normalization module include removal of motion artifacts and dividing the signal into multiple segments for further analysis.

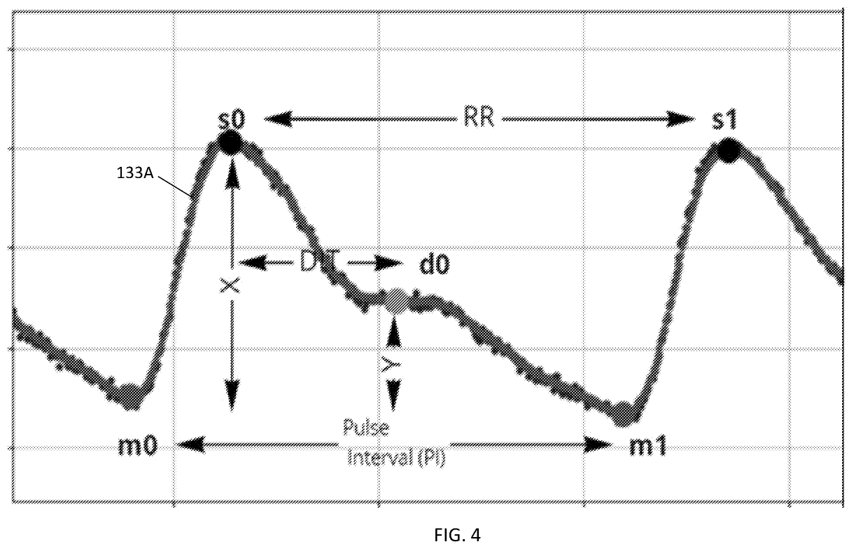

Reference is further made to FIG. 4 which is a graph, depicting an example of normalized PPG data or PPG signal 133A, according to some embodiments of the invention. According to some embodiments, normalization module 330 may normalize the at least one version of PPG signal (e.g., 210A, 131A, 132A) as elaborated herein.

Normalization module 133 may obtain (e.g., from artifact removal module 320) at least one portion or sequence of PPG signal 132A, that is devoid of artifacts. Normalization module 133 may segment the portion or sequence according to predefined locations (e.g., peaks, troughs) in PPG signal 132A, such that each segment corresponds to the number of heart-beat cycles, and may overlay the segments to produce a multi-cycle representation of PPG signal 132A.

As depicted by the blue dots in FIG. 4, the multi-cycle representation of PPG signal 132A may include a plurality of sampled PPG values 210A, pertaining to one or more heart-beat cycles. Normalization module 133 may then produce a normalized PPG signal 133A based on the plurality of overlaid sampled PPG values. For example, normalization module 330 may calculate an interpolation function of the plurality of overlaid sampled PPG values, to produce the normalized PPG signal 133A, as depicted by the continuous line in FIG. 4.

According to some embodiments, system 100 may include at least one machine learning (ML) based model 160 (e.g., 160A, 160B, 160C), trained to predict, or produce a prediction or classification of an inflammatory condition of a patient, based on at least one version (e.g., 210A, 131A, 132A, 133A) of incoming PPG signal 210A, as elaborated herein.

For example, system 100 may introduce or provide normalized PPG data 133A pertaining to a patient, to at least one ML-based model 160 as input. ML-based model 160 may subsequently calculate or predict, as commonly referred to in the art, a status or condition of the patient based on the input normalized PPG data element 133A.

Additionally, or alternatively, system 100 may include a feature extraction module 140, adapted to analyze the PPG signal (e.g., 133A) to produce or extract one or more PPG features 140A from normalized PPG signal or data element 133A, as elaborated herein. System 100 may introduce or provide the one or more PPG features 140A to the at least one ML-based model 160 as input. In other words, processor 180 may apply ML-based model 160 on normalized signal data element 133A, and may thus calculate, or predict a status or condition of inflammation 100A based on the one or more PPG features 140A.

According to some embodiments, feature extraction module 140 may extract one or more PPG features 140A such as the ones elaborated in Table 1, below:

TABLE 1
(where in the below table t represent the time and v represents the value)

| PPG Feature | | |
|---|---|---|
| 410 | Calculation | Comment |
| RR | T(s1) − T(s0) | Time difference between a first peak (marked s0) |
| | | and a subsequent peak (marked s1) of normalized |
| | | PPG data 133A, as depicted in FIG. 4. |
| PI | T(m1) − T(m0) | Time difference between a first trough (marked |
| | | m0) and a subsequent trough (marked m1) of |
| | | normalized PPG data 133A, as depicted in FIG. 4. |
| DIT | T(d0) − T(s0) | Time difference between the first peak, s0, and the |
| | | dicrotic notch marked d0, as depicted in FIG. 4. |
| RAI | (v(d0) − mean(v(m1) − | v indicates the value of the normalized PPG data at |
| | v(m0))/(v(s0) − | d0, m1, m0 and s0, as depicted in FIG. 4. |
| | mean(v(m1) − v(m0)) | |
| RDI | DIT/RR | Ratio DIT/RR |
| NRDI | (t(d0) − t(m0))/PI | Normalized Ratio DIT/RR |
| BoA | b/a (acc plot) | Ratio of points b and a; where a and b are points |
| | | along the second derivative wave of the PPG signal |
| MSL | (t(s0) − t(m0))/PI | Pulse interval normalized time difference between |
| | | s0 and m0, as depicted in FIG. 4 |
| LF | low frequency | |
| HF | high frequency | |
| LF/HF | Freq ratio | |
| DC | (v(m1) + v(m0))/2 | v indicates the value of the normalized PPG data at |
| | | m1 and m0 as depicted in FIG. 4 |
| AC | (v(s1) + v(s0)) − 2 − DC | V indicates the value of the normalized PPG data |
| | | at s1 and s0 as depicted in FIG. 4. |
| Perf_index | AC/DC | Ratio AC/DC |
| SpO2_R | Perf_index1/Perf_index2 | Ratio Peripheral oxygen saturation |

Additionally, or alternatively, feature extraction module 140 may analyze at least one physiological signal 220A and/or ambient signal 220B to produce one or more physiological, or ambient features 140B. For example, physiological signal 220A may include readings of an accelerometer, and physiological feature 140B may include a feature of the readings of an accelerometer, such as a maximal reading and/or average reading of the accelerometer. In another example, ambient signal 220B may include a reading of ambient lighting, and ambient features 140B may include a maximal reading of the ambient lighting and/or average reading of the ambient lighting. Additional physiological and/or ambient features may also be used.

According to some embodiments, processor 180 may apply ML-based model 160 on the one or more physiological and/or ambient features 140B, and may thus calculate, or predict a status or condition of inflammation 100A based on the one or more PPG features, and further based on the one or more physiological and/or ambient features 140B.

According to some embodiments, system 100 may receive, e.g., via a user interface (UI, such as input element 7 and/or output element 8 of FIG. 2) at least one biomarker data element 90, representing a value of a biomarker pertaining to the patient.

For example, the at least one biomarker data element 90 may include a value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value and/or a d-dimer value, blood transcriptomics, proteomics, metabolomics, microbiome and any combination thereof.

According to some embodiments, processor 180 may apply ML-based model 160 on the one or more biomarker data elements 90, to predict 110A the inflammatory condition of the patient further based on the one or more biomarker data elements 90.

According system 100 may receive, e.g., via a user interface (UI, such as input element 7 and/or output element 8 of FIG. 2) and/or from a computing device associated with a medical database 70 such as an Electronic Medical Record (EMR) or Electronic Health Record (EHR) at least one medical history data element 70A, representing information pertaining to medical history of the patient. Processor 180 may apply ML-based model 160 on the at least one medical history data element 70A to predict the inflammatory condition of the patient, further based on the at least one medical history data element 70A.

According to some embodiments, prediction or classification 100A of a patient's inflammatory condition or status may include, for example a trajectory of inflammatory flare-up. In such embodiment system 100 may produce, or present on a UI (e.g., elements element 7 and/or element 8 of FIG. 2) at least one notification 100A′ or warning regarding the patient's inflammatory condition (e.g., an upcoming flare-up) based on prediction or classification 100A.

Additionally, or alternatively, prediction or classification 100A of a patient's inflammatory condition or status may include, for example, a trajectory of inflammatory remission. In such embodiment system 100 may produce, or present on UI (e.g., 7 and/or 8) a notification 100A′ regarding the patient's inflammatory condition (e.g., inflammatory remission) based on prediction or classification 100A.

Additionally, or alternatively, prediction or classification 100A of a patient's inflammatory condition or status may include, for example a failure of anti-inflammatory treatment, and/or a suggestion for an anti-inflammatory treatment or drug. In such embodiment notification 100A′ may include a notification regarding failed, or recommended treatments, based on prediction or classification 100A.

According to some embodiments, the at least one ML based model 160 may be trained to predict a patient's inflammatory condition or status 100A based on a supervised training algorithm.

For example, the at least one ML based model 160 may receive a training dataset that may include normalized PPG signals or data element 133A, which may pertain to a plurality of patients and/or pertain to a plurality of samples taken from a single patient, or any combination thereof. Additionally, or alternatively, the at least one ML based model 160 may receive a training dataset of PPG features 140A as elaborated herein (e.g., in Table 1), which may pertain to a plurality of patients and/or pertain to a plurality of samples taken from a single patient, or any combination thereof.

Additionally, the at least one ML based model 160 may receive “ground-truth” labels or annotation 70 of inflammation condition or status corresponding to the training dataset. For example, the training dataset may be annotated by an expert physician, who may label or associate at least one normalized PPG signals or data element 133A and/or feature 140A as pertaining to a patient's inflammatory condition such as an inflammatory flare-up, an inflammatory remission, and the like. ML based model 160 may be trained according to these labels to predict the inflammatory condition based on normalized PPG signals or data elements 133A and/or features 140A/140B.

According to some embodiments, processor 180 may apply any suitable training algorithm as known in the art to train the at least one ML based model 160. For example, processor 180 may employ a gradient descent back-propagation algorithm, to train the at least one ML based model 160, based on the training set (e.g., PPG features 140A and/or normalized data 133A) and annotation data 80.

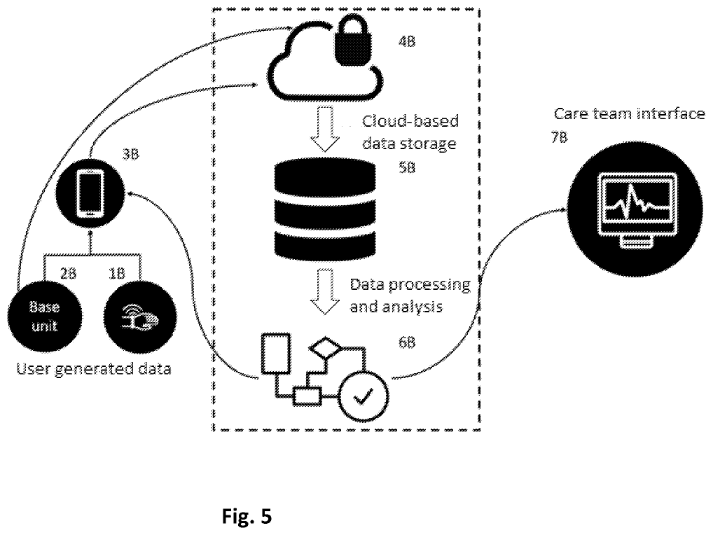

Reference is now made to FIG. 5, which is a schematic diagram representing an example of an implementation of a system (e.g., system 100 of FIG. 3a) for performing non-invasive assessment of inflammatory conditions in patients, according to some embodiments of the invention. According to some embodiments, component 1B may include a medical wearable/implantable sensor 20, or a sensory device having a multisensory array that may be configured to capture haemorheology parameters, including for example pulse wave velocity, physiological parameters and digital biomarkers. Data obtained from component 1B (e.g., measured by sensors 20) may be transferred (e.g., via Bluetooth, WiFi, etc.) to a computer 3B, such as a mobile phone device. Component 2B may be a base unit that may include a non-invasive monitoring unit for analysing samples (e.g., saliva samples) of the patient. For example, component 2B may utilize spectroscopy methods to analyse the (blood, saliva, urine) samples for therapeutics and biomarker levels. It should be noted that elements of component 2B may also be included in component 1B. In certain embodiments, and as illustrated in FIG. 5, data from components 1B, 2B and/or 3B may be uploaded and stored in a secured cloud-based database (denoted as components 4B and 5B). It is noted that digital biomarkers could be any selected from a group consisting of sleep patterns, steps count, indoor patterns, outdoor patterns, movement patterns, geolocation information, accelerometry information, actigraphy information, mobile use information and any combination thereof.

Component 6B may include a data processing and analytics platform, and may employ machine learning algorithms to generate predictive analytics insights regarding the clinical status of the patient. These predictions and/or clinical insights may be projected to the patient via component 3B and/or a computing device of a care team 7B.

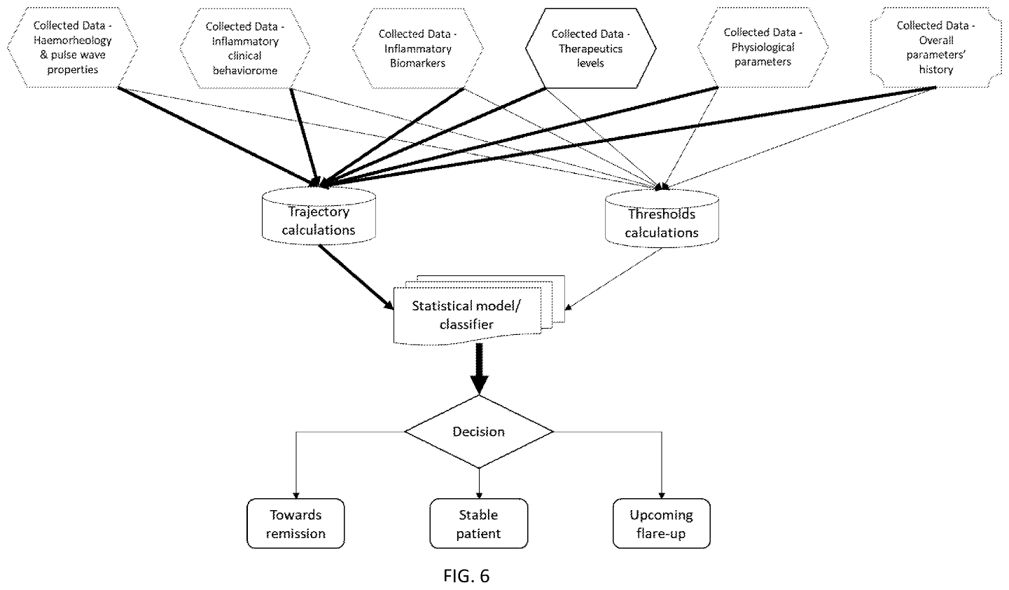

Reference is now made to FIG. 6, which is a schematic diagram, depicting a classifier algorithm that may be implemented by a system for non-invasive assessment of inflammatory conditions in patients according to some embodiments of the invention.

According to some embodiments, system 100 may monitor, manage and/or predict the progress of chronic diseases and treatment effectiveness. System 100 may be configured to collect and receive data, some continuously and some periodically. Then, the algorithm being used by system 100 may to generate a disease activity state based on predefined parameters, as well as on machine learning statistical models. The system can eventually create a prediction of a disease state trajectory, including for example whether a patient is stable or heading toward a remission or a flare-up. Accordingly, the system 100 can prompt a user (e.g., via a UI such as elements 7 and/or 8 of FIG. 2) for additional measurements from the different monitoring devices and/or trigger an alert or notify about the disease state.

In certain embodiments, the algorithm used in the system of the invention may include a predictor and/or classifier algorithm that may be based on machine learning tools and heuristic rules, such as age at disease onset, disease location, etc., to predict and determine the state of the disease's activity. Notably, the classifier may have two purposes: (a) to distinguish between patients with an active disease state and patients with remission; and (b) to identify subclinical inflammatory status and generate a prediction of an upcoming flare-up. These classifications and predictions may rely on continuous measurements and tracking changes from baseline patterns of each state.

According to some embodiments, the predictor/classifier may utilize any combination of the following as input parameters for generating the prediction and determination of the disease activity state:

### B. Blood Flow Rheology/Haemorheology:

blood flow rheological properties may be used to assess erythrocytes aggregation and plasma viscosity as reliable measures of acute phase inflammation. The blood flow rheological properties may be based on the tracking of changes from a predefined/pre-measured baseline. Such changes may be indicative of a subclinical inflammation before a flare-up occurs. Notably, it is a measurable tool for therapy efficiency in chronic inflammatory diseases.

The blood flow rheology/haemorheology algorithm may be used by system 100 to assess blood rheology properties by analyzing changes in the PPG signal obtained from illuminating blood vessels and skin of the subject at rest and after applying shear force on the blood in the blood vessels.

Reference is now made to FIG. 7, which is a graph depicting blood viscosity profile as a function of shear rate. As shown in FIG. 7, the present invention utilizes the fact that whole blood behaves as a non-Newtonian fluid and its viscosity is dependent on the amount shear rate exposed while sampling, as known in the art. Thus, the illumination and PPG signal acquisition may occur concomitantly with specified shear force applied that is measured by the accelerometer. It should be noted that applying shear force is performed by maneuvering said at least one location pertaining to the patient at at least one selected from a group consisting of predetermined speed, predetermined rhythm, for a predetermined period of time and any combination thereof.

According to some embodiments, controller 180 of FIG. 3a may control the optical sensor 210 and/or auxiliary sensor 220 (e.g., an accelerometer) to measure the mechanical effect and response of the blood flow as obtained from the collected PPG signal during acceleration of blood in the vessels. This approach may allow detection of changes in blood viscosity, blood flow and erythrocyte aggregation, all of which are markers for an active inflammation phase. Embodiments of the invention may thus include an improvement over currently available methods and systems of monitoring of haemorheology properties.

(B) Pulse Rate and (C) Pulse Wave Velocity Obtained from the PPG Signal:

It has been shown that in some chronic inflammatory diseases (such as IBD) arterial stiffness is associated with active inflammation. Increased arterial stiffness is dependent upon inflammation and reduced by immunomodulatory drugs. Specifically, during active disease/inflammation arterial stiffness is increased and may lead to measurable changes in pulse wave velocity (PWV). PWV is the speed of a pressure pulse propagating along the arterial wall and can be calculated from pulse transit time (PTT). PTT is the time between two pulse waves propagating on the same cardiac cycle from two arterial sites. PWV is the speed at which the forward pressure wave is transmitted from the aorta through the vascular tree, and is calculated by measuring the time taken for the arterial waveform to pass between two points a measured distance apart, and involves taking readings from the two sites simultaneously, or gating separate recordings to a fixed point in the cardiac cycle (e.g., the R-wave of the ECG, the PPG signal, pressure or flow signals, or a combination of both). As known in the art, the relationship between PWV and arterial stiffness may be the Moens Korteweg equation: PWV=√(E×h/2rp), where: E is Young's modulus of elasticity of wall material; h-wall thickness of vessel; r-inside radius of vessel; and p-density of blood.

According to some embodiments, system 100 and the algorithms used thereby may use continuous measurements of PWV, and may track changes in arterial stiffness to assess and predict, together with other parameters, the disease state trajectory. Accordingly, arterial stiffness is non-invasively assessed according to pulse wave velocity measurements obtained from the PPG signal. Moreover, some studies indicate that changes in radial pulse wave velocity may occur with high blood viscosity.

According to some embodiments, system 100 may determine PWV by PPG signal or with a combination of PPG and Electrocardiogram (ECG) signals.

The term “Electrocardiogram” refers hereinafter to a record of the heart's electrical activity.

By employing a pair of sensors 20 (e.g., 2 PPG sensors or 1 PPG sensor and one ECG sensor), the amount of time taken by the pulse wave to traverse an arterial segment can be determined and used to calculate the PWV, according to the following equation: PWV=L/PTT, where L is the distance between the two measurement points; and PTT is the transit time of the pulse wave (e.g., the time delay between the two signals).

### (D) Biomarker Analysis & (E) Therapeutics Levels:

Therapeutics and biomarkers levels help to define the patient's clinical status and disease activity status. Therapeutics levels are important to determine therapy efficiency and its impact on disease activity. Biomarkers levels (such as inflammatory biomarkers) are important to determine the disease activity status. Such data may be obtained from measurements of saliva, blood and/or urine samples using spectroscopy analysis and/or by non-invasive optical measurements of samples thereof.

According to this embodiment of the present invention analysis of blood or saliva samples will be used to track biomarkers or therapeutics levels in addition to the non-invasive PPG measurements at home.

### (F) Inflammatory Clinical Behaviorome:

The term behaviorome refers hereinafter to capturing, analysis and interpretation of human behavior as a determinant of health. Thus, behaviorome may be used herein to refer to a set of digital markers (e.g., step counts, when staying at home vs. outdoor patterns) that can be collected, and may reflect a patient's status, such as fatigue that is correlated with increased inflammation.

This is a collection of activities and behaviors that can be collected and analyzed using the sensors array 20 and passive data collection. This sub-algorithm may analyze all the collected data and may utilize machine learning tools and heuristic rules to identify patterns and hidden patterns that correlate with disease activity. In certain embodiments, the data may be collected from various sensors 20 that are able to capture daily activities such as: motion sensors, geolocators, UV sensors, heart rate sensor, body temperature sensors, humidity sensor, ambient light sensor etc. Accordingly, in some embodiments, the activity types and behaviors may be collected and analyzed to generate the “inflammatory clinical behaviorome”.

According to some embodiments of the invention, system 100 may utilize any combination of the above mentioned sub-algorithms/variables. The combination of sub-algorithms/variables as well as their importance and relevance (i.e., impact) varies between different diseases; between patients with the same condition/disease; and between different stages of disease/condition within a specific patient (e.g., between flare-up and remission).

According to some embodiments, the activity types and behaviors include, but not limited to overall all activity patterns; daily steps counter patterns; body temperature patterns; sleep patterns; indoor and outdoor duration patterns; eating habits/appetite patterns; number of daily visits in the restrooms, etc., and any combination thereof.

According to some embodiments, the classifier/predictor algorithm may be based on machine learning tools and heuristic rules (such as age at disease onset, disease location, etc.) to predict and/or determine the disease activity state. The classifier/predictor algorithm may also utilize other algorithms as listed below as input parameters for generating the prediction and to determine the disease activity state.

The classifier/predictor algorithm may (a) to distinguish between patients with active disease state versus patients on remission; and (b) identify subclinical inflammatory status and generate a prediction of an upcoming flare-up. These classifications and predictions may rely on continuous measurements and tracking changes from baseline patterns at each state.

FIG. 6 depicts the classifier algorithm according to certain embodiments of the invention. The classifier algorithm may be based on supervised or non-supervised machine learning or heuristic rules. The algorithm may perform data analysis for a variety of patient's data: haemorheology, PWV, ECG, inflammatory clinical behaviorome, inflammatory biomarkers, physiological parameters (such as body temperature) and therapeutics levels. The algorithm may classify the disease state and alert for a required intervention for a particular patient by continuous comparison to overall parameters history and disease trajectory calculations. The classification can be rules based on thresholds and statistical model. Statistical models may include machine learning algorithms such as: neural networks logistic regression, decision tree, decision forest, K-means, SVM and others. These algorithms/measurements may be used as input parameters for the main classifier/predictor algorithm.

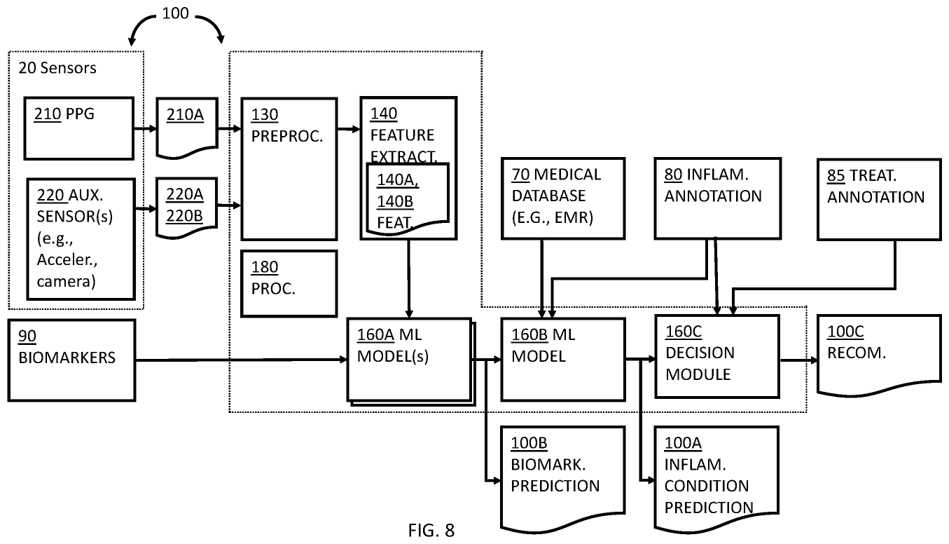

Reference is now made to FIG. 8, which is a block diagram depicting another example of system 100 for performing non-invasive assessment of inflammatory conditions in patients, according to some embodiments of the invention. It may be appreciated that components of system 100 of FIG. 8 may be substantially similar to those of system 100 of FIG. 3a, and their description will not be repeated here for the purpose of brevity.

As elaborated herein (e.g., in relation to FIG. 3a), processor 180 may apply one or more ML models 160 on one or more features (e.g., PPG features 140A and/or physiological or ambient features 140B) to predict an inflammatory condition or status 100A of a patient. As shown in FIG. 8, system 100 may include a plurality of ML models 160, depicted as elements 160A, 160B and 160C.

According to some embodiments, processor 180 may apply at least one first ML model 160A on one or more features (e.g., PPG features 140A and/or physiological or ambient features 140B), to predict at least one biomarker value 100B. processor 180 may subsequently apply at least one second ML model 160B on said predicted biomarker value 100B, to predict the inflammatory condition 100A of the patient.

For example, processor 180 may apply at least one first ML model 160A on PPG features 140A and/or physiological or ambient features 140B, to predict at least one biomarker value 100B.

The at least one predicted biomarker value 100B may include, for example a value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value and/or a d-dimer value, blood transcriptomics, proteomics, metabolomics, microbiome and any combination thereof.

Processor 180 may subsequently apply at least one second ML model 160B on one or more of the predicted biomarker values 100B, to produce a prediction or classification 100A of an inflammatory condition of the patient.

According to some embodiments, the at least one ML based model 160A may be trained to predict a value of at least one specific inflammation biomarker value 100B based on a supervised training algorithm.

For example, the at least one ML based model 160A may receive an annotated training dataset of normalized PPG signals or data elements 133A. The training dataset may pertain to a plurality of subjects (e.g., patients) and/or pertain to a plurality of samples taken from to a single subject or patient, or any combination thereof. Additionally, or alternatively, the training dataset may include annotated PPG features 140A as elaborated herein (e.g., in Table 1) and/or annotated features 140B, which may pertain to a plurality of patients and/or pertain to a plurality of samples taken from a single patient, or any combination thereof.

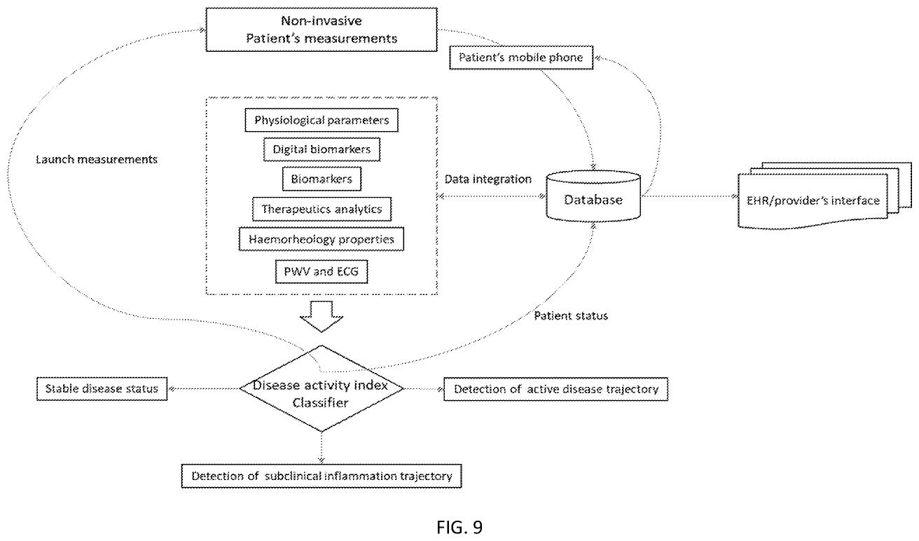

The term “annotated” may be used in this context to indicate that elements of the training dataset may be associated with “ground-truth” labels of inflammation biomarker values, corresponding to the training dataset. For example, and as elaborated herein (e.g., in relation to FIGS. 9-12), ML based model 160A may receive data representing measured values of inflammation biomarkers 90 (e.g., PLT, ESR, CRP, Fecal calprotecin and the like) from a blood sample, as measured by laboratory tests. ML based model 160A may utilize the measured values of inflammation biomarkers 90 as labels or supervisory data for training.

According to some embodiments, processor 180 may apply any suitable training algorithm as known in the art to train the at least one ML based model 160A. For example, processor 180 may employ a gradient descent back-propagation algorithm, to train the at least one ML based model 160A, based on the training set (e.g., features 140A, features 140B, and/or normalized data 133A) and biomarkers label data 90.

According to some embodiments, ML model 160B may be trained to produce a prediction or classification 100A of a patient's inflammatory condition based on a supervised training algorithm.

For example, ML model 160B may receive an annotated training dataset of predicted biomarker values 100B, where one or more (e.g., each) predicted biomarker value 100B may be associated with a label or annotation of an inflammatory condition 80. In a subsequent stage of inference, processor 180 may apply or infer ML based model 160B on predicted biomarker values 100B to produce a prediction or classification 100A of a patient's inflammatory condition.

Additionally, or alternatively, processor 180 may produce a notification (e.g., a message, such as an email message) that may include one or more predicted biomarker values 100B. Processor 180 may subsequently transmit the notification of predicted biomarker values 100B to at least one computing device (e.g., elements 3B, 7B of FIG. 5), such as a caregiver's computing device.

According to some embodiments, system 100 may include a decision module 160C. Decision module 160C may be configured to receive input data such as prediction or classification 100A of a patient's inflammatory condition, and/or one or more predicted inflammation biomarkers 100B. Decision module 160C may subsequently produce a recommendation of treatment 100C, based on the received input data.

According to some embodiments, decision module 160C may be, or may include an ML-based model, that may be trained to produce recommendation 100C based on a supervised training algorithm.

For example, ML model 160C may receive an annotated training dataset that may include a plurality of classifications 100A of a patient's inflammatory condition. The training dataset of classifications 100A may be annotated in a sense that one or more (e.g., each) classifications 100A may be associated with a “ground-truth” annotation or label of treatment 85. Treatment annotation 85 may include, for example a recommended treatment or drug that may be prescribed by an expert (e.g., a physician) for treating a corresponding predicted inflammatory condition 100A of a patient.

Reference is now made to FIG. 9 illustrating a system for non-invasive assessment of inflammatory conditions in patients, according to some embodiments of the invention.

As seen in the Figure and as described above, noninvasive patient's measurements are taken including (physiological parameters, digital biomarker, biomarker, haemorheology properties PWC and ECG). Thereafter all data is integrated to provide a diseases activity index to detect whether the disease is stable or to provide a detection of the active disease trajectory. Such analysis will also be provided to the patient's mobile phone or to a care giver thereof and/or electronic health record, EHR.

It should be pointed out that although the above disclosure relates to the analysis of a PPG signal (as a stand alone or in combination with auxiliary sensors (e.g., accelerometer), it is within the scope of the present invention to provide a system and device that provide indication of at least one inflammatory status of a patient, comprising steps of
illuminating at least one location pertaining to the patient with at least one optical light beam characterized by at least one wavelength;
detecting at least one signal; said signal is at least a portion of at least one selected from a group consisting of the transmitted light beam, the absorbed light beam, the reflected light beam and any combination thereof; and,
analyzing said at least a portion of said signal as a function of time thereby indicating said inflammatory status of said patient;
   wherein said step of analyzing at least a portion of said signal additionally comprising step of: (a) extracting at least one feature; (b) analyzing at least one trend of said at least one feature as a function of time; and, (c) correlating said at least one trend with at least one biomarker indicative of said at least one inflammatory status.

It further within the scope of the present invention where detecting is performed by at least one sensor selected from a group consisting of photodiode and/or laser light source.

It is further within the scope of the present invention, where the wavelength/s used is/are as follows:

For photodiode:
Visible light 400-700 nm
And IR from 1 mm to 700 nm

For the laser light source:
the whole diode laser spectrum can be used in medical applications of diode lasers cover, starting from 200 nm ultraviolet and violet DLs used for sterilization and some surgery applications, through photodynamic therapy (PDT) in the visible wavelength range at 630-690 nm, to longer wavelengths.

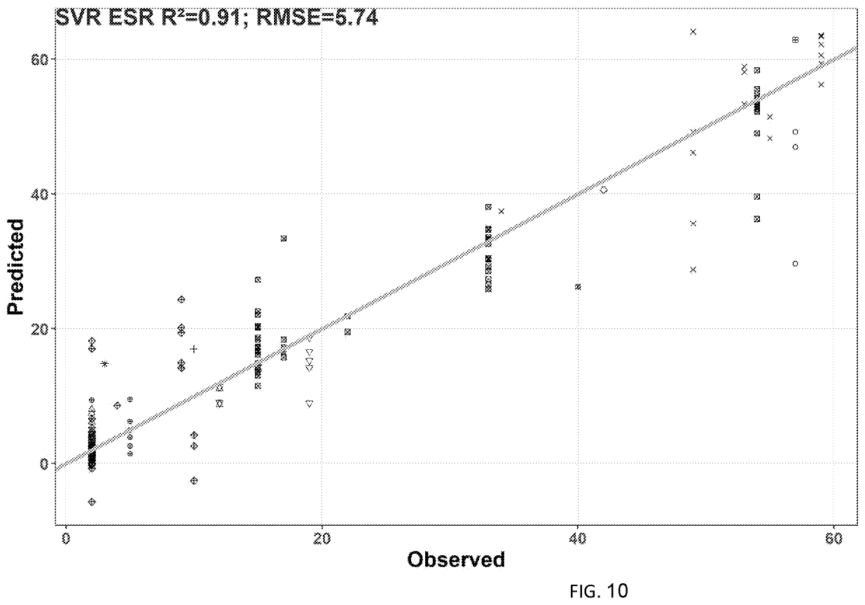

Reference is now made to FIG. 10 which is a point plot graph, depicting an example of measured values vs. predicted values of Erythrocyte Sedimentation Rate (ESR), pertaining to a plurality of patients, according to some embodiments of the invention. As shown in FIG. 10, Observed vs. Predicted values based on the non-invasive measurements of ESR values according to the present invention. The coefficient of determination (commonly referred to as “R squared”) between the measured and predicted values is 0.91. Different shapes represent different patients. The model was based on ˜26 patients (depending on label and sensor measurement) with quality-controlled data points and labels. The model was based on nonlinear (′rbf) SVR with Train/Test ratio of 0.75/0.25 (i.e., The model was trained on 75% from all records and the plots show results of predictions on the remaining 25%).

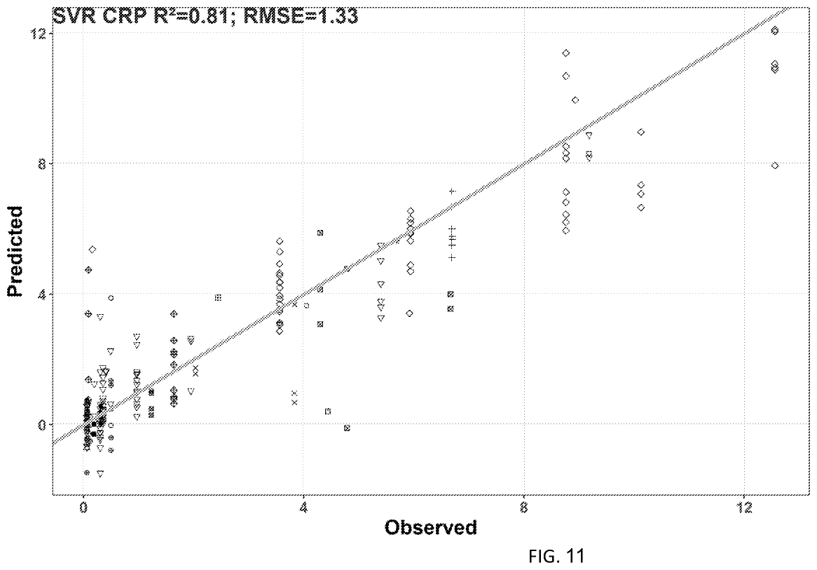

Reference is now made to FIG. 11 which is a point plot graph, depicting an example of measured values vs. predicted values of C-reactive protein (CRP), pertaining to a plurality of patients, according to some embodiments of the invention. As shown in FIG. 11, Observed vs. Predicted values based on the non-invasive measurements of CRP values according to the present invention. The coefficient of determination (“R squared”) between the measured and predicted values is 0.81. The model was based on ˜26 patients (depending on label and sensor measurement) with quality-controlled data points and labels. The model was based on non-linear (‘rbf’) SVR with Train/Test ratio of 0.75/0.25

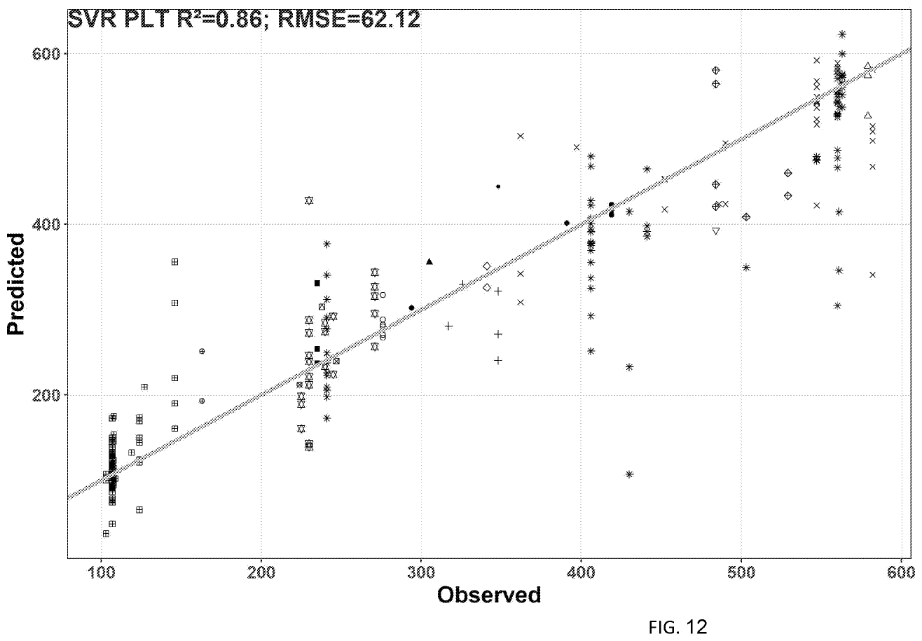

Reference is now made to FIG. 12 which is a point plot graph, depicting an example of measured values vs. predicted values of Platelet count (PLT), pertaining to a plurality of patients, according to some embodiments of the invention. As shown in FIG. 11, Observed vs. Predicted values based on the non-invasive measurements of PLT values of the present invention. The coefficient of determination (“R squared”) between the measured and predicted values is 0.86. The model was based on ˜26 patients (depending on label and sensor measurement) with quality-controlled data points and labels. The model was based on non-linear (‘rbf’) SVR with Train/Test ratio of 0.75/0.25.

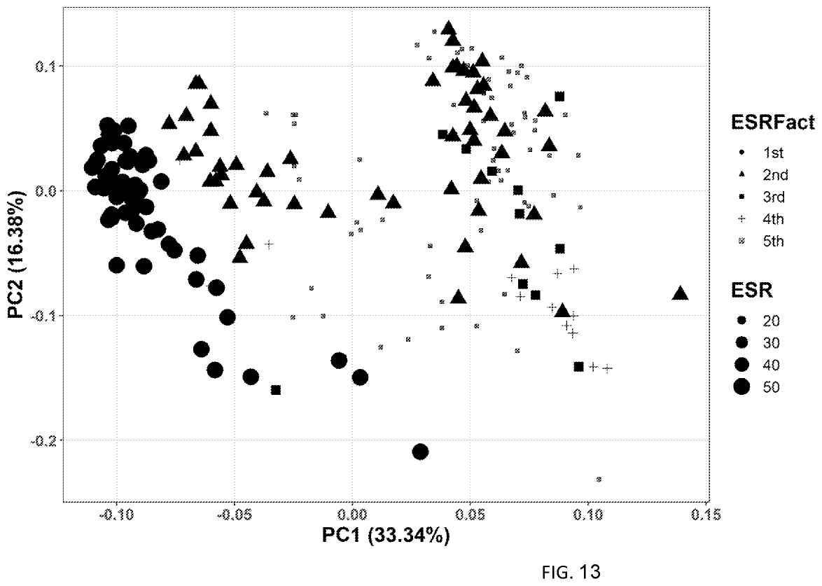

Reference is now made to FIG. 13 illustrating a principal component analysis (PCA) of a single patient. Dimensionality reduction using PCA performed to investigate the variability between PPG signals and how they correlate to ESR levels. Different shapes represent different days while size represents the ESR level. Consistent personal PPG signal capture is found to be indicative of ESR measurements. Good separation was observed between low, medium high ESR.

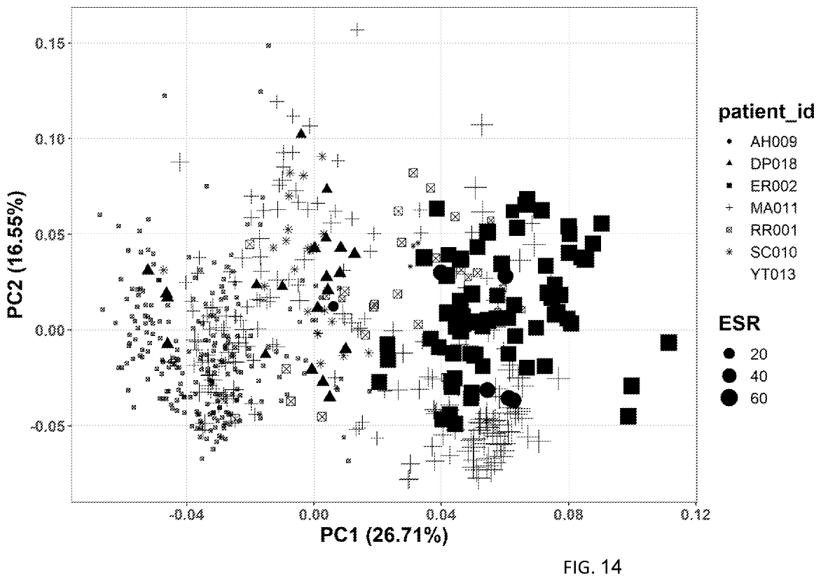

Reference is now made to FIG. 14 illustrating a principal component analysis (PCA) of multiple patients. Dimensionality reduction using PCA performed to investigate the variability between PPG signals and how they correlate to ESR levels. Different shapes represent different patients while size represents the ESR level A similar trend suggests that the variance between PPG signals is associated with both patients and ESR levels. Good separation between low, medium high ESR. Common PPG features are associated with ESR correlation.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Although certain embodiments have been illustrated and described herein, it will be appreciated by those of ordinary skill in the art that a wide variety of alternative or equivalent embodiments or implementations, calculated to achieve the same or similar purposes, may be substituted for the embodiments illustrated and described herein without departing from the scope of the present invention. Those of skill in the art will readily appreciate that embodiments in accordance with the present invention may be implemented in a very wide variety of ways. This application is intended to cover any and all adaptations and/or variations of the embodiments discussed herein.

The terms and expressions which have been employed in the foregoing specification are used therein as terms of description and not of limitation, and there is no intention, in the use of such terms and expressions, to exclude equivalents of the features shown and/or described or portions thereof, it being recognized that the scope of the invention is defined and limited only by the claims that follow.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

1. A method of indicating at least one inflammatory status of a patient, comprising steps of:
illuminating at least one location pertaining to the patient with at least one optical light beam characterized by at least one wavelength;
detecting at least one signal; said signal is at least a portion of at least one selected from a group consisting of the transmitted light beam, the absorbed light beam, the reflected light beam and any combination thereof; and
analyzing said at least a portion of said signal as a function of time thereby indicating said inflammatory status of said patient;
wherein said step of analyzing at least a portion of said signal additionally comprising step of:
(a) extracting at least one feature;
(b) analyzing at least one trend of said at least one feature as a function of time; and
(c) correlating said at least one trend with at least one biomarker indicative of said at least one inflammatory status.

2. The method of claim 1, wherein at least one step selected from a group consisting of illuminating, detecting, analyzing and any combination thereof is performed continuously.

3. The method of claim 1, wherein said step of detecting is performed by at least one sensor selected from a group consisting of photodiode, laser light source and any combination thereof.

4. The method of claim 1, wherein said step of illuminating at least one optical light beam is performed in a manner selected from pulsed, continues and any combination thereof.

5. The method of claim 1, wherein said at least one wavelength is in the range of about 200 nm to about 800 nm and/or 1 mm to 700 nm.

6. The method of any one of claims 1 to 5, wherein said steps of (a) illuminating at least one location pertaining to the patient with at least one optical light beam characterized by at least one wavelength; and step of (b) detecting at least a portion of at least one selected from a group consisting of the transmitted light beam, the absorbed light beam, the reflected light beam and any combination thereof; is performed by at least one photoplethysmogram (PPG).

7. The method of any one of claims 1 to 6, wherein said step of analyzing said at least a portion of said signal is performed by analyzing at least one photoplethysmography (PPG) signal from at least one location pertaining to the patient by means of said at least one photoplethysmogram (PPG).

8. The method of any one of claims 1 to 7, wherein said step of illuminating at least one location pertaining to the patient with at least one optical light beam characterized by at least one wavelength is performed by at least one optical source selected from a group consisting of photodiode, laser light source and any combination thereof.

9. The method of any one of claims 1 to 8, wherein said step of analyzing said at least a portion of said signal as a function of time, additionally comprising step of analyzing the intensity of said signal as a function of time.

10. The method of any one of claims 1 to 9, wherein said step of analyzing said at least a portion said signal as a function of time, additionally comprising step of providing at least one reference signal as a function of time.

11. The method of claim 10, wherein said at least one reference signal is at least one selected from a group consisting of the transmitted light beam, the absorbed light beam, the reflected light beam and any combination thereof as a function of time, of at least one selected from a group consisting of said patient with a known inflammatory status, of at least one group of patients of with known inflammatory status, an average of at least one group of patients of with known inflammatory status, and any combination thereof.

12. The method of claim 11, wherein said known inflammatory status is selected from a group consisting of a flare-up, a remission, a healthy pattern and any combination thereof.

13. The method of any one of claims 10 to 12, wherein said step of analyzing said at least a portion of said signal as a function of time, additionally comprising step of comparing and identifying deviations of the same with said at least one reference signal as a function of time.

14. The method of any one of claims 1 to 13, additionally comprising step of providing at least one baseline pattern of said PPG signal for each of said inflammatory status, such that a deviation above a predetermined threshold from said at least one baseline pattern indicates a change of said inflammatory status.

15. The method of claim 14, wherein said baseline pattern represents at least one selected from a group consisting if a flare-up, a remission, a healthy pattern and any combination thereof.

16. The method of any one of claims 1 to 15, additionally comprising step of providing the patient's medical history.

17. The method of any one of claims 1 to 16, wherein said at least one PPG signal is obtained by at least one wearable device, a patch placed on said patient's skin, noncontact measurement or any combination thereof.

18. The method of any one of claims 1 to 17, wherein said at least one signal is obtained by an implantable device.

19. The method of any one of claims 1 to 18, wherein said step of analysis is performed by at least one artificial intelligence modality, machine-learning (ML) modality, neural network, deep learning, artificial neural network and any combination thereof.

20. The method of any one of claims 1 to 19, wherein said at least one feature is selected from a group consisting of time difference between a first peak and a subsequent peak, RR, time difference between a first trough and a subsequent trough, PI, time difference between the first peak and the dicrotic notch, DIT, ratio DIT/RR, normalized ratio DIT/RR, BoA feature, MSL feature, low frequency sum, LF, high frequency sum, HF, DC feature, AC feature, peripheral index feature, ratio of peripheral indices and any combination thereof.

21. The method of any one of claims 1 of 20, wherein said inflammatory status is selected from a group consisting of: a trajectory of inflammatory flare-up, a trajectory of inflammatory remission, a failure of anti-inflammatory treatment and any combination thereof.

22. The method of any one of claims 1 to 21, further comprising step of producing at least one notification pertaining to said inflammatory status.

23. The method of claim 22, wherein said notification includes information selected from a group consisting of said inflammatory status, suggested treatment, a change of treatment corresponding to said inflammatory status and any combination thereof.

24. The method of claim 23, wherein said suggested treatment includes at least one selected from a group consisting of administration of at least one pharmacological agent.

25. The method of claim 23, wherein said at least one pharmacological agent is selected from a group consisting of anti-inflammatory drugs, steroids, immunosuppressives, anti-inflammatory monoclonal antibodies, anti-inflammatory molecules and any combination thereof.

26. The method of any one of claims 1 to 25, wherein said inflammatory status pertains to at least one disease selected from a group consisting of immune mediated diseases; said immune mediated diseases being selected from a group consisting of Inflammatory Bowel Diseases (IBD) including ulcerative colitis (UC) and Crohn's disease (CD), Rheumatological diseases, Rheumatoid Arthritis (RA), Psoriatic Arthritis (PsA), Spondyloarthritis, (SpA) Psoriasis, Chronic Obstructive Pulmonary Disease (COPD), Asthma, Systemic Lupus Erythematosus (SLE), Multiple Sclerosis (MS), Vasculitis, malignant diseases, cardiovascular diseases, Immune-Mediated Heart Diseases, Infection-Related Immune-Mediated Diseases, and any combination thereof.

27. The method of any one of claims 1 to 26, wherein said at least one PPG signal is obtained by a device comprising fixation element adapted to apply pressure on said device such that said device is stabilized and maintained pressed against a predetermined location on the patient's skin, from which said at least one PPG signal is obtained.

28. The method of claim 27, wherein said predetermined location on the patient's skin is selected from a group consisting of at least one finger, arm, forearm, wrist, ear, leg, ankle, scalp, abdominal, thoracic areas and any combination thereof.

29. The method of claim 27 or 28, wherein said fixation element is selected from a group consisting of at least one electro-mechanical element, at least one inflating balloon, a spring-based mechanism, a shape-memory alloy-based mechanism and any combination thereof.

30. The method of any one of claims 27 to 29, wherein said fixation element is adapted to apply pressure on said device such that said device is maintained pressed against a predetermined location on the patient's skin, from which said at least one PPG signal is obtained, in a cyclic manner.

31. The method of any one of claims 27 to 30, wherein said cyclic manner comprising steps of:
a. immediately before said step of receiving at least one PPG signal, applying pressure and pressing said device against said predetermined location on the patient's skin by said fixation element;
b. immediately after said step of receiving at least one PPG signal, releasing said pressure applied by said fixation element onto said device.

32. The method of any one of claims 1 to 31, additionally comprising step of receiving at least one biomarker level pertaining to said patient.

33. The method of claim 32, wherein said step of receiving at least one biomarker level pertaining to said patient is provided by measurement selected from a group consisting of spectroscopy analysis, non-invasive optical measurements and any combination thereof of samples selected from a group consisting of saliva, blood, urine and any combination thereof.

34. The method of claim 32 or 33, wherein said biomarker data includes at least one selected from a group consisting of value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value, d-dimer value and blood transcriptomics, proteomics, metabolomics, microbiome and combination thereof.

35. The method of any one of claims 1 to 34, additionally comprising step of obtaining at least one haemorheology parameter associated with blood flow haemorheology.

36. The method of claim 35, wherein said at least one blood flow haemorheology parameter is selected from a group consisting of pulse wave velocity, physiological parameters and any combination thereof.

37. The method of any one of claims 1 to 36, additionally comprising step of obtaining at least one blood flow rheological parameter associated with blood flow rheological properties.

38. The method of claim 37, wherein said at least one blood flow rheological parameter is adapted to indicate at least one selected from a group consisting of erythrocytes aggregability and deformability, blood flow, plasma viscosity, vascular resistance, hematocrit and any combination thereof.

39. The method of claim 37 or 38, wherein said at least one blood flow rheological parameter indicates said inflammatory status.

40. The method of any one of claims 37 to 39, additionally comprising step of providing at least one baseline pattern of at least one blood flow rheological parameter for each of said inflammatory status, such that a deviation above a predetermined threshold from said at least one baseline pattern indicates a change of said inflammatory status.

41. The method of any one of claims 1 to 40, additionally comprising steps of
a. momentarily, for a predetermined period of time, reducing blood flow from reaching said at least one location pertaining to said patient; thereby said at least one PPG signal is attenuated;
b. after said predetermined period of time, enabling said blood flow to reach said at least one location pertaining to said patient; thereby said at least one PPG signal is regained;
c. analyzing at least one feature of the attenuated signal versus the regained signal;
wherein said inflammatory status is provided based on said analysis.

42. The method of claim 41, wherein said step of analyzing additionally comprising step of measuring the amount of time needed for said at least one PPG signal to regain; wherein said inflammatory status is provided based on said amount of time needed for said at least one PPG signal to regain.

43. The method of claim 41, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

44. The method of claim 41, wherein said step of momentarily reducing blood flow is performed by applying pressure on at least one predetermined location.

45. The method of any one of claims 41 to 44, wherein said step of momentarily reducing blood flow is performed by means of at least one selected from a group consisting of a cuff, an inflatable cuff, at least one electro-mechanical element, at least one inflating balloon, a spring-based mechanism, a shape-memory alloy-based mechanism and any combination thereof, at least partially encircling said at least one predetermined location.

46. The method of any one of claims 41 to 45, wherein said steps of (a) momentarily reducing blood flow; and, said step of (b) enabling said blood flow to reach said at least one location pertaining to said patient, are synchronized with said step of detecting at least one signal; such that said signal is synchronized and detected immediately after said step of momentarily reducing blood flow; and immediately after said step of enabling said blood flow to reach said at least one location pertaining to said patient.

47. The method of any one of claims 41 to 46, additionally comprising steps of:
a. applying, for a predetermined period of time, at least one vibration on said at least second location, substantially different from said at least one location; thereby said at least one PPG signal is changed;
b. after said predetermined period of time, removing said applied vibration; thereby said at least one PPG signal is regained;
c. analyzing at least one feature of at least one selected from a group consisting of the changed signal, the changed signal versus the regained signal and any combination thereof;
wherein said inflammatory status is provided based on said analysis.

48. The method of any one of claims 41 to 47, wherein said feature is selected from a group consisting of the amount of time needed for said at least one PPG signal to regain; the advancement rate of said vibrations from said at least one second location to said at least one location, the intensity thereof as a function of time, and any combination thereof.

49. The method of any one of claims 41 to 48, wherein said at least one predetermined location is selected from a group consisting of armpit, forearm, finger, leg, ankle, wrist, ear and any combination thereof.

50. The method of any one of claims 1 to 49, additionally comprising step of inducing acceleration of blood in said at least one location pertaining to said patient where said at least one PPG signal is received.

51. The method of claim 50, wherein said step of inducing acceleration of blood in said at least one location is performed by applying shear force on the same.

52. The method of claim 50 or 51, additionally comprising step of analyzing changes in said at least one PPG signal before and after said step of applying shear force on blood in said at least one location pertaining to said patient thereby indicating said inflammatory status of said patient.

53. The method of any one of claims 50 to 52, wherein said step of applying shear force is measured by communicating at least one accelerometer with said at least one location pertaining to said patient.

54. The method of any one of claims 50 to 53, wherein said applying shear force is performed by maneuvering said at least one location pertaining to the patient at least one selected from a group consisting of predetermined speed, predetermined rhythm, for a predetermined period of time and any combination thereof.

55. The method of any one of claims 50 to 54, additionally comprising steps of
a. inducing acceleration of blood in said at least one location is performed by applying shear force on the same; thereby elevating the PPG signal;
b. analyzing at least one feature of the attenuated signal versus the regained signal;
wherein said inflammatory status is provided based on said analysis.

56. The method of claim 55, additionally comprising step of obtaining said PPG signal only when said step of applying shear force of said at least one location is above a predetermined threshold.

57. The method of claim 55, wherein said step of analyzing additionally comprising step of measuring the amount of time needed for said at least one PPG signal to regain; wherein said inflammatory status is provided based on said amount of time needed for said at least one PPG signal to regain.

58. The method of claim 55, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

59. The method of claim 55, wherein said amount of time needed for said at least one PPG signal to regain is indicative of at least one blood flow rheological parameter selected from a group consisting of erythrocytes aggregability and deformability, blood flow, plasma viscosity, vascular resistance, hematocrit and any combination thereof.

60. The method of any one of claims 1 to 59, additionally comprising step of receiving at least one signal, by at least one sensor, pertaining to movement of said patient.

61. The method of claim 60, wherein said at least one sensor is selected from a group consisting of accelerometer, a camera, a microphone, a step counter, a sleep quality sensor and any combination thereof.

62. The method of any one of claims 1 to 61, additionally comprising step of receiving at least one signal, by at least one sensor, selected from a group consisting of ambient light sensor, a thermometer, and any combination thereof.

63. The method of any one of claims 1 to 62, additionally comprising step of measuring changes in pulse wave velocity, PWV.

64. The method of claim 63, wherein said changes in said PWV are indicative of arterial stiffness.

65. The method of claim 63 or 64, wherein increase in said arterial stiffness is indicative of inflammation and reduction in said arterial stiffness is indicative of inflammatory remission.

66. The method of any one of claims 63 to 65, wherein said step of measuring changes in said PWV is performed by calculating the pulse transit time, PTT, between at least two pulse waves propagating on the same cardiac cycle from two arterial sites.

67. The method of any one of claims 63 to 66, wherein PWV=√(E×h/2rp), where E=Young's modulus of elasticity of wall material; h=wall thickness of vessel; r=inside radius of vessel; and ρ=density of blood.

68. The method of any one of claims 63 to 67, wherein said at least two pulse waves propagating on the same cardiac cycle from two arterial sites are selected from a group consisting of a pair of PPG signals, a pair of PPG and Electrocardiogram (ECG) signals.

69. The method of any one of claims 1 to 68, additionally comprising step of receiving behaviorome data pertaining to said patient.

70. The method of claim 69, wherein said behaviorome data is obtained from sensors selected from a group consisting of indoor and outdoor step counts, motion sensors, geolocators, sleep patterns; indoor and outdoor duration patterns; eating habits/appetite patterns; number of daily visits in the restrooms, motion sensors, geolocators, UV sensors, heart rate sensor, daily steps counter patterns, body temperature sensors, humidity sensor, ambient light sensor, movement patterns, geolocation information, accelerometry information, actigraphy information, mobile use information and any combination thereof.

71. The method of any one of claims 1 to 70, additionally comprising step of analyzing said at least one PPG signal to thereby provide a prediction of the future inflammatory status of said patient.

72. A system for indicating inflammatory status in a patient, comprising:
a monitoring device, adapted to obtain from at least one location pertaining to said patient at least one signal; said signal is at least a portion of at least one selected from a group consisting of a transmitted light beam, an absorbed light beam, a reflected light beam and any combination thereof from at least one optical light beam illuminated on said at least one location;
a processor in communication with said monitoring device, adapted to analyze said at least a portion of at least a portion of at least one selected from a group consisting of the transmitted light beam, the absorbed light beam, the reflected light beam and any combination thereof as a function of time by (a) extracting at least one feature; (b) analyzing at least one trend of said at least one feature as a function of time; and, (c) correlating said at least one trend with at least one biomarker indicative of said at least one inflammatory status, to thereby indicate said inflammatory status of said patient.

73. The system of claim 72, wherein said at least one optical light beam is characterized by at least one wavelength.

74. The system of claim 73, wherein said at least one wavelength is in the range of about 200 nm to about 800 nm and/or 1 mm to 700 nm.

75. The system of claim 72, additionally comprising at least one optical source selected from a group consisting of photodiode, laser light source, and any combination thereof, adapted to illuminate said at least one location pertaining to the patient with at least one optical light beam.

76. The system of claim 75, wherein said at least one optical source is adapted to illuminate said at least one optical light beam in a manner selected from pulsed, continues and any combination thereof.

77. The system of claim 72, additionally comprising at least one photodiode adapted to detect at least a portion of said signal as a function of time.

78. The system of claim 72, wherein said monitoring device is at least one photoplethysmogram (PPG).

79. The system of any one of claims 72 to 78, wherein processor is adapted to analyze one photoplethysmography (PPG) signal from at least one location pertaining to the patient by means of said at least one photoplethysmogram (PPG).

80. The system of claim 72, wherein processor is adapted to analyze the intensity of at least a portion of said signal as a function of time.

81. The system of any one of claims 72 to 80, wherein processor is adapted to compare at least a portion of said signal as a function of time with said at least one reference signal as a function of time.

82. The system of claim 81, wherein said at least one reference signal is at least one selected from a group consisting of the transmitted light bean, the absorbed light beam, the reflected light beam and any combination thereof as a function of time, of at least one selected from a group consisting of said patient with a known inflammatory status, at least one group of patients of with known inflammatory status an average of at least one group of patients of with known inflammatory status, and any combination thereof.

83. The system of claim 82, wherein said known inflammatory status is selected from a group consisting of a flare-up, a remission, a healthy pattern and any combination thereof.

84. The system of any one of claims 72 to 83, wherein said processor is either in direct or indirect physical communication with said monitoring device.

85. The system of any one of claims 72 to 84, wherein said processor is in wirelessly communication with said monitoring device.

86. The system of any one of claims 72 to 85, wherein said monitoring device is adapted to continuously provide said at least one PPG signal.

87. The system of any one of claims 72 to 86, wherein said processor is adapted to provide at least one baseline pattern of said PPG signal for each of said inflammatory status, such that a deviation above a predetermined threshold from said at least one baseline pattern indicates a change of said inflammatory status.

88. The system of any one of claims 72 to 87, wherein said processor performs said analysis by at least one artificial intelligence modality, machine-learning (ML) modality, neural network, deep learning, artificial neural network and any combination thereof.

89. The system of any one of claims 72 to 88, wherein said processor is adapted to extract from said PPG signal over time at least one feature; said at least one feature is selected from a group consisting of time difference between a first peak and a subsequent peak, RR, time difference between a first trough and a subsequent trough, PI, time difference between the first peak and the dicrotic notch, DIT, ratio DIT/RR, normalized ratio DIT/RR, BoA feature, MSL feature, low frequency sum, LF, high frequency sum, HF, DC feature, AC feature, peripheral index feature, ratio of peripheral indices and any combination thereof.

90. The system of any one of claims 72 to 89, wherein said inflammatory status is selected from a group consisting of: a trajectory of inflammatory flare-up, a trajectory of inflammatory remission, a failure of anti-inflammatory treatment and any combination thereof.

91. The system of any one of claims 72 to 90, additionally comprising at least one notification system adapted to provide notification pertaining to said inflammatory status.

92. The system of claim 91, wherein said notification includes information selected from a group consisting of said inflammatory status, suggested treatment, a change of treatment corresponding to said inflammatory status and any combination thereof.

93. The system of claim 92, wherein said suggested treatment includes at least one selected from a group consisting of administration of at least one pharmacological agent.

94. The system of claim 93, wherein said at least one pharmacological agent is selected from a group consisting of anti-inflammatory drugs, steroids, immunosuppressives, anti-inflammatory monoclonal antibodies, anti-inflammatory molecules and any combination thereof.

95. The system of any one of claims 72 to 94, wherein said inflammatory status pertains to at least one disease selected from a group consisting of immune mediated diseases; said immune mediated diseases being selected from a group consisting of Inflammatory Bowel Diseases (IBD) including Ulcerative Colitis (UC) and Crohn's disease (CD), Rheumatological diseases, Rheumatoid Arthritis (RA), Psoriatic Arthritis (PsA), Spondyloarthritis (SpA) Psoriasis, Chronic Obstructive Pulmonary Disease (COPD), Asthma, Systemic Lupus Erythematosus (SLE), Multiple Sclerosis (MS), Vasculitis, malignant diseases, cardiovascular diseases, Immune-Mediated Heart Diseases, Infection-Related Immune-Mediated Diseases and any combination thereof.

96. The system of any one of claims 72 to 95, wherein said monitoring device additionally comprising fixation element adapted to apply pressure on said monitoring device such that said monitoring device is stabilized and maintained pressed against a predetermined location on the patient's skin, from which said PPG signal is obtained.

97. The system of claim 96, wherein said predetermined location on the patient's skin is selected from a group consisting of at least one finger, arm, forearm, wrist, ear, leg, ankle, scalp, abdominal, thoracic areas and any combination thereof.

98. The system of claim 96 or 97, wherein said fixation element is selected from a group consisting of at least one electro-mechanical element, at least one inflating balloon, a spring-based mechanism, a shape-memory alloy-based mechanism and any combination thereof.

99. The system of any one of claims 96 to 98, wherein said fixation element is adapted to apply pressure on said monitoring device such that said monitoring device is stabilized and maintained pressed against a predetermined location on the patient's skin, from which said PPG signal is obtained, in a cyclic manner.

100. The system of any one of claims 96 to 99, wherein said cyclic manner comprising steps of:
a. immediately before said step of receiving at least one PPG signal, applying pressure and pressing said device against said predetermined location on the patient's skin by said fixation element;
b. immediately after said step of receiving at least one PPG signal, releasing said pressure applied by said fixation element onto said device.

101. The system of any one of claims 72 to 100, wherein said monitoring device additionally comprising means of receiving at least one biomarker level pertaining to said patient.

102. The system of claim 101, wherein said means of receiving at least one biomarker level pertaining to said patient is selected from a group consisting of measurement selected from a group consisting of spectroscopy analysis, non-invasive optical measurements and any combination thereof of samples selected from a group consisting of saliva, blood, urine and any combination thereof.

103. The system of claim 101 or 102, wherein said biomarker data includes at least one selected from a group consisting of value of a platelet count, an erythrocyte sedimentation rate value, a c-reactive protein concentration value, a fecal calprotectin concentration value, a blood viscosity value, a perinuclear antineutrophil cytoplasmic antibodies' value, an anti-Saccharomyces cerevisiae antibodies value, a lactoferrin value, a lipocalin-2 value, a serum albumin value, a serum amyloid A value, a ferritin value, a fibronectin value, an orosomucoid, α1-acid glycoprotein value, a plasminogen value, IL-1, IL-4, IL-5, and/or IL-10 value, TNF-α, IFN-α, IL-2, IL-6, IL-8, IL-12, IL-23, IL-23R and/or LIF-1 value, a rheumatoid factor value, an anti-cyclic citrullinated peptide value, an IL-12p40 value, an interferon alpha value, IL-15, CCL3, CCL11 and/or CXCL13 value, a calgranulin value, a VEGF value, an angiopotietin-2 value, d-dimer value blood transcriptomics, proteomics, metabolomics, microbiome and any combination thereof.

104. The system of any one of claims 72 to 103, wherein said monitoring device additionally comprising means of receiving at least one haemorheology parameter associated with blood flow haemorheology.

105. The system of claim 104, wherein said at least one blood flow haemorheology parameter is selected from a group consisting of pulse wave velocity, physiological parameters and any combination thereof.

106. The system of any one of claims 72 to 105, wherein said monitoring device additionally comprising means of obtaining at least one blood flow rheological parameter associated with blood flow rheological properties.

107. The system of claim 106, wherein said at least one blood flow rheological parameter is adapted to indicate at least one selected from a group consisting of erythrocytes aggregability and deformability, blood flow, plasma viscosity, vascular resistance, hematocrit and any combination thereof.

108. The system of claim 106 or 107, wherein said at least one blood flow rheological parameter indicates said inflammatory status.

109. The system of any one of claims 106 to 108, wherein said processor is adapted to provide at least one baseline pattern of at least one blood flow rheological parameter for each of said inflammatory status, such that a deviation above a predetermined threshold from said at least one baseline pattern indicates a change of said inflammatory status.

110. The system of any one of claims 72 to 109, additionally comprising means adapted to
a. momentarily, for a predetermined period of time, reduce blood flow from reaching said at least one location pertaining to said patient; thereby said at least one PPG signal is attenuated;
b. after said predetermined period of time, enable said blood flow to reach said at least one location pertaining to said patient; thereby said at least one PPG signal is regained.

111. The system of claim 110, wherein said processor is adapted to analyze at least one feature of the attenuated signal versus the regained signal; wherein said inflammatory status is provided based on said analysis.

112. The system of claim 110, wherein said feature is selected from a group consisting of the amount of time needed for said at least one PPG signal to regain, the rate at which said at least one PPG signal is regained, the intensity of said attenuated PPG signal, the integral of the signal as a function of time, the derivative of the signal as a function of time and any combination thereof.

113. The system of claim 110, wherein said processor is adapted to measure the amount of time needed for said at least one PPG signal to regain; wherein said inflammatory status is provided based on said amount of time needed for said at least one PPG signal to regain.

114. The system of claim 110, wherein said means of momentarily reducing blood flow is performed by applying pressure on at least one predetermined location.

115. The system of claim 110, wherein said step of momentarily reducing blood flow is performed by means of at least one selected from a group consisting of a cuff, an inflatable cuff, at least one electro-mechanical element, at least one inflating balloon, a spring-based mechanism, a shape-memory alloy-based mechanism and any combination thereof, at least partially encircling said at least one predetermined location.

116. The system of any one of claims 110 to 115, additionally comprising at least one vibrating element adapted to apply vibration to at least one second location, substantially different from said at least one location.

117. The system of any one of claims 110 to 116, additionally comprising means adapted to:
a. apply, for a predetermined period of time, at least one vibration on said at least one second location; thereby said at least one signal is changed;
b. after said predetermined period of time, remove said applied vibration; thereby said at least one signal is regained;
c. analyze at least one feature of at least one selected from a group consisting of the changed signal, the changed signal versus the regained signal and any combination thereof;
wherein said inflammatory status is provided based on said analysis.

118. The system of any one of claims 110 to 117, wherein said feature is selected from a group consisting of the amount of time needed for said at least one signal to regain; the advancement rate of said vibrations from said at least one second location to said at least one location, the intensity thereof as a function of time, and any combination thereof.

119. The system of any one of claims 110 to 118, wherein said at least one predetermined location is selected from a group consisting of armpit, forearm, finger, leg, ankle, wrist, ear, and any combination thereof.

120. The system of any one of claims 72 to 119, additionally comprising means of inducing acceleration of blood in said at least one location pertaining to said patient where said at least one signal is received.

121. The system of claim 120, wherein said means of inducing acceleration of blood in said at least one location is performed by applying shear force on the same.

122. The system of claim 121, wherein said monitoring device is adapted to obtain said PPG signal only when said applying shear force of said at least one location is above a predetermined threshold.

123. The system of any one of claims 120 to 122, wherein said processor is adapted to analyze changes in said at least one PPG signal before and after said applying shear force on blood in said at least one location pertaining to said patient; and, thereby to indicate said inflammatory status of said patient.

124. The system of any one of claims 120 to 123, additionally comprising means adapted to induce acceleration of blood in said at least one location is performed by applying shear force on the same; thereby elevating the PPG signal.

125. The system of any one of claims 120 to 124, wherein said applying shear force is performed by maneuvering said at least one location pertaining to the patient at at least one selected from a group consisting of predetermined speed, predetermined rhythm, for a predetermined period of time and any combination thereof.

126. The system of claim 125, wherein said processor is adapted to measure the amount of time needed for said at least one PPG signal to regain; wherein said inflammatory status is provided based on said amount of time needed for said at least one PPG signal to regain.

127. The system of claim 126, wherein said amount of time needed for said at least one PPG signal to regain is indicative of at least one blood flow rheological parameter selected from a group consisting of erythrocytes aggregability and deformability, blood flow, plasma viscosity, vascular resistance, hematocrit and any combination thereof.

128. The system of any one of claims 72 to 127, wherein said monitoring device additionally comprising at least one sensor adapted to provide data pertaining to movement of said patient.

129. The system of claim 128, wherein said at least one sensor is selected from a group consisting of accelerometer, a camera, a microphone, a step counter, a sleep quality sensor and any combination thereof.

130. The system of any one of claims 72 to 129, wherein said monitoring device additionally comprising at least one sensor selected from a group consisting of ambient light sensor, a thermometer, and any combination thereof.

131. The system of any one of claims 72 to 130, wherein said processor adapted to measure changes in pulse wave velocity, PWV.

132. The system of claim 131, wherein said changes in said PWV are indicative of arterial stiffness.

133. The system of claim 131 or 132, wherein increase in said arterial stiffness is indicative of inflammation and reduction in said arterial stiffness is indicative of inflammatory remission.

134. The system of any one of claims 131 to 133, wherein said changes in said PWV is performed by calculating the pulse transit time, PTT, between at least two pulse waves propagating on the same cardiac cycle from two arterial sites.

135. The system of any one of claims 131 to 134, wherein PWV=√(E×h/2rp), where: E=Young's modulus of elasticity of wall material; h=wall thickness of vessel; r=inside radius of vessel; and ρ=density of blood.

136. The system of any one of claims 131 to 135, wherein said at least two pulse waves propagating on the same cardiac cycle from two arterial sites are selected from a group consisting of a pair of PPG signals, a pair of PPG and Electrocardiogram (ECG) signals.

137. The system of any one of claims 72 to 136, wherein said monitoring device additionally comprising at least one sensor adapted to provide behaviorome data pertaining to said patient.

138. The system of claim 137, wherein said at least one sensor is selected from a group consisting of indoor and outdoor step counts, motion sensors, geolocators, sleep patterns; indoor and outdoor duration patterns; eating habits/appetite patterns; numbers of daily visits in the restrooms, motion sensors, geolocators, UV sensors, heart rate sensor, daily steps counter patterns, body temperature sensors, humidity sensor, ambient light sensor, movement patterns, geolocation information, accelerometry information, actigraphy information, mobile use information and any combination thereof.

139. The system of any one of claims 72 to 138, wherein the monitoring device is a non-invasive wearable device.

140. The system of any one of claims 72 to 139, wherein the monitoring device is a patch device.

141. The system of any one of claims 72 to 140, wherein the monitoring device is implantable.
